(19) United States
(12) Patent Application Publication
(10) Pub. No.: US 2008/0090827 A1
(43) Pub. Date: Apr. 17, 2008

(54) Compounds With Mixed Pde-Inhibitory and Beta-Adrenergic Antagonist or Partial Agonist Activity For Treatment of Heart Failure

(75) Inventors: Malcolm George Taylor, Didcot (GB); Burkhard Klenke, Didcot (GB); Peter D. Suzdak, Towson, MD (US); Reza Mazhari, Towson, MD (US)

Correspondence Address: SEED INTELLECTUAL PROPERTY LAW GROUP PLLC, 701 FIFTH AVE, SUITE 5400, SEATTLE, WA, 98104, US

(73) Assignee: Artesian Therapeutics, Inc., Gaithersburg, MD (US)

(21) Appl. No.: 11/791,893

(22) PCT Filed: Nov. 8, 2005
(86) PCT No.: PCT/US05/40586

§ 371 Date: Dec. 10, 2007

Related U.S. Application Data
(60) Provisional application No. 60/631,598, filed on Nov. 30, 2004.

(51) Int. Cl. A61K 31/50; A61K 31/444; A61P 25/06; A61P 25/08; A61P 27/02; A61P 9/00; C07D 237/04; C07D 401/04

(52) U.S. Cl. 514/247; 514/334; 544/239; 546/257

(57) ABSTRACT

This invention provides compounds that possess inhibitory activity against β-adrenergic receptors and phosphodiesterase PDE, including phosphodiesterase 3 (PDE3). This invention further provides pharmaceutical compositions comprising such compounds; methods of using such compounds for treating cardiovascular disease, stroke, epilepsy, ophthalmic disorder or migraine; and methods of preparing pharmaceutical compositions and compounds that possess inhibitory activity against β-adrenergic receptors and PDE.

### BACKGROUND OF THE INVENTION

1. Field of the Invention

The present invention is directed to novel compounds possessing both PDE-inhibitory and β-adrenergic receptor agonist activities.

2. Description of the Related Art

Congestive heart failure affects an estimated 4.8 million Americans with over 400,000 new cases diagnosed each year. Despite incremental advances in drug therapy, the prognosis for patients with advanced heart failure remains poor with annual mortality exceeding 40 percent. Although heart transplantation is an effective therapy for patients with advanced heart failure, less than 2,200 heart transplants are performed annually due to a limited supply of donor organs. Recent analyses indicate that further increases in the incidence and prevalence of advanced heart failure are likely, highlighting the pressing need for novel and effective therapeutic strategies.

During heart failure, there is an alteration of calcium homeostasis, including impaired sarcoplasmic reticulum calcium re-uptake, increased basal (diastolic) calcium levels, decreased peak (systolic) calcium and reduced rate of calcium transients, resulting in a decreased force of contraction and a slowing of relaxation. The end results of these abnormalities in calcium homeostasis are depressed contractile function (decreased contractility and cardiac output), impaired ventricular relaxation, and myocyte loss via ischemia and/or apoptosis-related mechanisms. Disregulation of calcium homeostasis has also been implicated in a number of other disease states, including stroke, epilepsy, ophthalmic disorders, and migraine.

Beta-adrenergic blocking agents are common therapy for patients with mild to moderate chronic heart failure (CHF). Some patients on β-blockers may subsequently decompensate, however, and would need acute treatment with a positive inotropic agent. Phosphodiesterase inhibitors (PDEI), such as milrinone or enoximone, retain their full hemodynamic effects in the face of beta-blockade, because the site of PDEI action (cAMP) is downstream of the β-adrenergic receptor, and because β-antagonism reverses receptor pathway desensitization changes, which are detrimental to phosphodiesterase inhibitor response.

### BRIEF SUMMARY OF THE INVENTION

This invention provides compounds that possess inhibitory activity against β-adrenergic receptors and phosphodiesterase PDE, including phosphodiesterase 3 (PDE3). This invention further provides pharmaceutical compositions comprising such compounds; methods of using such compounds for treating cardiovascular disease, stroke, epilepsy, ophthalmic disorder or migraine; and methods of preparing pharmaceutical compositions and compounds that possess inhibitory activity against β-adrenergic receptors and PDE.

### BRIEF DESCRIPTION OF THE SEVERAL VIEWS OF THE DRAWING(S)

FIG. 1 is a graph depicting the percent increase in left ventricular contractility upon treatment of anesthetized rabbits with various doses of Compound 13.

### DETAILED DESCRIPTION OF THE INVENTION

The present invention is based upon the development of novel dual-pharmacophore small molecule compounds that possess both phosphodiesterase and β-adrenergic receptor inhibitory activity. The compounds of the present invention retain the positive attributes of β-adrenergic receptor antagonism without producing depression of cardiovascular function by simultaneously antagonizing both the β-adrenergic receptor and phosphodiesterase-3. As described herein, compounds of the present invention were found to augment cellular contractility in the absence of isoproterenol, and elicit a potent β-blocking effect antagonizing the effects of isoproterenol, in an in vivo animal model. Thus, these compounds are able to normalize β-adrenergic receptor signaling while maintaining normal myocardial contractility and, therefore, represent a new class of drugs for the treatment of heart failure and hypertension.

In certain embodiments, the compounds of the present invention comprise a phosphodiesterase inhibitor tethered to a β-adrenergic receptor inhibitor by a linker. In one embodiment, the linker is substantially cleaved in vivo, to produce degradant metabolites that are biologically active. In other embodiments, the linker is substantially stable in vivo, i.e., it is not cleaved or not cleaved to a substantial degree, and the compound possesses both phosphodiesterase inhibitor and β-adrenergic receptor inhibitor activities. In either embodiment, the compounds of the present invention provide advantageous pharmacokinetics over therapies that involve the concurrent treatment of a patient with separate phosphodiesterase inhibitors and β-adrenergic blockers, in part due to the ability of the dual pharmacophore to deliver both active agents to the same location, tissue, or cell, thereby ensuring that the same cells and tissues adversely affected by treatment with the β-adrenergic blocker are provided with positive inotropic support.

### DEFINITIONS

“Alkyl radicals” refer to radicals of branched and unbranched saturated hydrocarbon chains comprising a designated number of carbon atoms. For example, C₁-C₉ alkyl radicals designates radicals of straight and branched hydrocarbon chains containing from 1 to 9 carbon atoms and includes all isomers. In some embodiments of the present invention, the alkyl radicals are C₁-C₁₂ radicals, and in other embodiments they are C₁-C₆ radicals. In yet other embodiments, the alkyl radicals are chosen from methyl, ethyl, propyl, iso-propyl, butyl, iso-butyl, tert-butyl, n-pentyl, and n-hexyl.

“Alkenyl radicals” refer to radicals of branched and unbranched unsaturated hydrocarbon chains comprising a designated number of carbon atoms. For example, C₂-C₉ alkenyl radicals designates radicals of straight and branched hydrocarbon chains containing from 2 to 9 carbon atoms having at least one double bond and includes all isomers. In some embodiments of the present invention, the alkenyl radicals are C₂-C₆ and in others thy are C₃-C₉. In yet other embodiments, the alkenyl radicals are chosen from ethenyl, propenyl, iso-propenyl, butenyl, iso-butenyl, tert-butenyl, n-pentenyl, and n-hexenyl.

“Alkynyl radicals” refer to radicals of branched and unbranched unsaturated hydrocarbon chains comprising a designated number of carbon atoms containing a triple bond between at least two carbon atoms and includes all isomers. For example, a C₂-C₉ alkynyl designates straight and branched hydrocarbon chains containing from 2 to 9 carbon atoms having at least one triple bond and includes all isomers. In some embodiments of the present invention, the alkynyl radicals are C₂-C₆, and in others they are C₃-C₉. In some embodiments, the alkynyl radicals are chosen from ethynyl, propynyl, iso-propynyl, butynyl, iso-butynyl, tert-butynyl, and pentynyl, and hexynyl.

“Alkylene radicals” refer to bivalent radicals of alkanes and includes all isomers.

“Alkenylene radicals” refer to bivalent radicals of alkenes having at least one double bond and includes all isomers.

“Alkynylene radicals” refer to bivalent radicals of alkynes having a triple bond between at least two carbon atoms and includes all isomers.

“Cycloalkyl radicals” refer to mono- or poly-cyclic alkyl radicals having a designated number of carbon atoms. For example, C₃-C₈ cycloalkyl radicals designate radicals of straight and branched hydrocarbon chains containing from 3 to 8 carbon atoms and includes all isomers. In some embodiments of the present invention, the cycloalkyl radicals are C₅-C₈ radicals. In yet other embodiments, the cycloalkyl radicals are chosen from methylcyclopropane, ethylcyclopropane, propylcyclopropane, butylcyclopropane, pentylcyclopropane, methylcyclobutane, ethylcyclobutane, propylcyclobutane, butylcyclobutane, methylcyclopentane, ethylcyclopentane, propylcyclopentane, methylcyclohexane, ethylcyclohexane, cyclobutyl, cyclopentyl, cyclohexyl, cycloheptyl, and cyclooctyl.

“Cycloalkenyl radicals” refer to mono- or poly-cyclic alkyl radicals having a designated number of carbon atoms and at least one double bond. For example, C₃-C₈ cycloalkenyl radicals designate radicals of straight and branched hydrocarbon chains containing from 3 to 8 carbon atoms, having at least one double bond and includes all isomers. In some embodiments of the present invention, the cycloalkenyl radicals are C₅-C₈ radicals. In yet other embodiments, the cycloalkenyl radicals are chosen from methylcyclopentene, ethylcyclopentene, propylcyclopentene, methylcyclohexene, ethylcyclohexene, cyclopentenyl, cyclohexenyl, cycloheptenyl, and cyclooctenyl.

“Cycloalkynyl radicals” refer to cyclic alkyl radicals having a designated number of carbon atoms and at least one triple bond. For example, C₃-C₈ cycloalkynyl radicals designates radicals of straight and branched hydrocarbon chains containing from 3 to 8 carbon atoms, having at least one triple bond and includes all isomers. In some embodiments of the present invention, the cycloalkynyl radicals are C₅-C₈ radicals. In yet other embodiments, the alkyl radicals are chosen from methylcyclohexyne, ethylcyclohexyne, cyclohexynyl, cycloheptynyl, and cyclooctynyl.

“Cycloalkylene radical” refers to a bivalent cycloalkyl radical.

“Heterocycloalkylene radical” refers to a bivalent saturated mono- or poly-cyclic alkyl radical, in which one or more carbon atoms is/are replaced by one or more heteroatom(s), such as nitrogen, phosphorous, oxygen, sulfur, silicon, germanium, selenium and/or boron. In some embodiments, the heteroatom(s) is/are nitrogen. Nonlimiting examples of heterocycloalkylene radicals include piperazinyl, morpholinyl, tetrahydropyranyl, tetrahydrofuranyl, piperidinyl and pyrrolidinyl.

“Alkylthio” refers to a sulfur substituted alkyl radical.

“Alkoxy” refers to the group —OR, wherein R is an alkyl radical as defined above. In some embodiments of the present invention, R is chosen from branched and unbranched saturated hydrocarbon chains containing from 1 to 9 carbon atoms. In some embodiments, R is chosen from alkyl radicals like C₁-C₆ and C₃-C₉. In yet other embodiments, the alkyl radicals are chosen from methyl, ethyl, propyl, iso-propyl, butyl, iso-butyl, tert-butyl, n-pentyl, and n-hexyl.

“Aryl” refers to aromatic, hydrocarbon cyclic moieties having one or more closed rings. For example, aryl may be chosen from C₆ to C₂₄ and from C₁₀ to C₁₈ aromatic hydrocarbon cyclic moieties. In some embodiments, aryl is chosen from phenyls, benzyls, naphthyls, anthracenyls, phenanthracenyls, and biphenyls. In yet other embodiments, aryl is chosen from phenyl, benzyl, naphthyl, anthracenyl, phenanthracenyl, and biphenyl.

“Heteroaryl” refers to aromatic, cyclic moieties having one or more closed rings with one or more heteroatoms in at least one of the rings. For example, heteroaryl may be chosen from 5- to 7-membered monocyclic and bicyclic or 7- to 14-membered bicyclic ring systems containing carbon atoms and 1, 2, 3 or 4 heteroatoms independently chosen from a nitrogen atom, an oxygen atom, and a sulfur atom. In some embodiments, heteroaryl radicals are chosen from pyrroles, furanyls, thiophenes, pyridines and isoxazoles. In yet other embodiments, heteroaryl is chosen from radicals of furans, benzofurans, benzothiophenes, oxazoles, thiazoles, benzopyrans and carbazoles.

“Halo radicals” refers to fluoro, chloro, bromo, and iodo radicals.

“Substituted phenyl” refers to phenyls that are substituted with one or more substituents. For example, the substituents may be chosen from C₁-C₆ alkyl radicals, C₂-C₆ alkenyl radicals, C₂-C₆ alkynyl radicals, C₁-C₆ alkoxy radicals, C₂-C₆ alkenyloxy radicals, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, trifluoromethoxy, iodosyl, iodyl, phosphino, phosphinyl, phospho, phosphono, arsino, selanyl, disilanyl, siloxy, silyl, silylene and carbocyclic and heterocyclic moieties.

“Effective amount” refers to the amount sufficient to produce a desired effect. For example, an effective amount for treating heart failure is an amount sufficient to treat heart failure; an effective amount for treating chronic heart failure is an amount sufficient to treat chronic heart failure; an effective amount for inhibiting PDE is an amount sufficient to inhibit PDE; an effective amount for inhibiting PDE 3 is an amount sufficient to inhibit PDE 3; and an effective amount for inhibiting β-adrenergic receptors is an amount sufficient to inhibit the β-adrenergic receptors.

“Metabolite” refers to a substance produced by metabolism or by a metabolic process.

“Pharmaceutically-acceptable carrier” refers to pharmaceutically-acceptable materials, compositions, and vehicles, such as liquid and solid fillers, diluents, excipients, and solvent encapsulating materials, involved in carrying or transporting the subject compound from one organ, or portion of the body, to another organ, or portion of the body. Each carrier is “acceptable” in the sense of being compatible with the other ingredients of the formulation and being suitable for use with the patient. A pharmaceutically-acceptable carrier may be active or inactive with respect to the patient. In some embodiments, pharmaceutically-acceptable carrier are chosen from: (1) sugars, such as lactose, glucose and sucrose; (2) starches, such as corn starch and potato starch; (3) cellulose band its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable equivalent” includes, without limitation, pharmaceutically acceptable salts, hydrates, solvates, metabolites, prodrugs, and isosteres. Many pharmaceutically acceptable equivalents are expected to have the same or similar in vitro or in vivo activity as the compounds of the invention.

“Pharmaceutically acceptable salt” refers to acid and base salts of the inventive compounds, which salts are neither biologically nor otherwise undesirable. In some embodiments, the salts can be formed with acids, and in some embodiments the salts can be formed form acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. In some embodiments, the salts can be formed from base salts, and in other embodiments the salts can be formed from ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds, using conventional methodology described, for instance, in BURGER'S MEDICINAL CHEMISTRY AND DRUG CHEMISTRY (5th ed.), volume 1 at pages 172-178, 949-982 (1995) (the disclosure of which is incorporated herein by reference).

“Isosteres” refer to elements, functional groups, substituents, molecules or ions having different molecular formulae but exhibiting similar or identical physical properties. For example, tetrazole is an isostere of carboxylic acid because it mimics the properties of carboxylic acid even though they have different molecular formulae. Typically, two isosteric molecules have similar or identical volumes and shapes. Ideally, isosteric compounds should be isomorphic and able to co-crystallize. Other physical properties that isosteric compounds often share include boiling point, density, viscosity and thermal conductivity. However, certain properties may be different, such as dipolar moments, polarity, polarization, size and shape, since the external orbitals may be hybridized differently. The term “isosteres” encompasses “bioisosteres,” which, in addition to their physical similarities, share some biological properties. Typically, bioisosteres interact with the same recognition site or produce broadly similar biological effects.

“Stereoisomers” are isomers that differ only in the arrangement of the atoms in space.

“Enantiomers” are stereoisomers that are non-superimposable mirror images of one another.

“Enantiomer-enriched” is a phrase that denotes a mixture in which one enantiomer predominates.

“Animal” refers to a living organism having sensation and the power of voluntary movement, and which requires for its existence oxygen and organic food. Examples include, without limitation, members of the human, equine, porcine, bovine, murine, canine, and feline species. In the case of a human, an “animal” may also be referred to as a “patient.” “Mammal” refers to a warm-blooded vertebrate animal.

“Treating” refers to: (i) preventing a disease, disorder or condition from occurring in an animal that may be predisposed to the disease, disorder and/or condition but has not yet been diagnosed as having it; (ii) inhibiting a disease, disorder or condition, i.e., arresting its development; and/or (iii) relieving a disease, disorder or condition, i.e., causing regression of the disease, disorder and/or condition.

“Heart failure” refers to the pathophysiologic state in which an abnormality of cardiac function is responsible for the failure of the heart to pump blood at a rate commensurate with the requirements of the metabolizing tissues.

“Congestive heart failure” refers to heart failure that results in the development of congestion and edema in the metabolizing tissues.

“Hypertension” refers to elevation of systemic blood pressure.

“SA/AV node disturbance” refers to an abnormal or irregular conduction and/or rhythm associated with the sinoatrial (SA) node and/or the atrioventricular (AV) node.

“Arrhythmia” refers to abnormal heart rhythm. In arrhythmia, the heartbeats may be too slow, too fast, too irregular or too early. Examples of arrhythmia include, without limitation, bradycardia, fibrillation (atrial or ventricular) and premature contraction.

“Hypertrophic subaortic stenosis” refers to enlargement of the heart muscle due to pressure overload in the left ventricle resulting from partial blockage of the aorta.

“Angina” refers to chest pain associated with partial or complete occlusion of one or more coronary arteries in the heart.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### Compounds

This invention provides compounds of formula (I)
β-(Ar)ₙ-(L)ₘ-X (I)
or a pharmaceutically acceptable equivalent, an isomer or a mixture of isomers thereof, wherein:

m is chosen from 0 and 1;

n is chosen from 0 and 1;

β is chosen from a 2-amino-1-hydroxyeth-1-yl radical, N-substituted-2-amino-1-hydroxyeth-1-yl radicals, N—N-disubstituted-2-amino-1-hydroxyeth-1-yl radicals, a 3-amino-2-hydroxypropoxy radical, N-substituted-3-amino-2-hydroxypropoxy radicals, and N—N-disubstituted-3-amino-2-hydroxypropoxy radicals;

Ar is chosen from aryl radicals and heteroaryl radicals, which aryl and heteroaryl radicals are unsubstituted or substituted with independently substituent(s) chosen from R₂, R₃, and R₄;

R₂, R₃, and R₄ are independently chosen from C₁-C₈ alkyl radicals, C₃-C₈ cycloalkyl radicals, C₂-C₈ alkenyl radicals, C₃-C₈ cycloalkenyl radicals, C₂-C₈ alkynyl radicals, C₃-C₈ cycloalkynyl radicals, C₁-C₄ alkylthio groups, C₁-C₄ alkoxy groups, halo radicals, a nitro group, a cyano group, a trifluoromethyl group, a trifluoroethyl group, a pentafluoroethyl group, a trifluoromethoxy group, —NR₅R₆ groups, acylaminoalkyl radicals, —NHSO₂R₁ groups and —NHCONHR₁ groups, wherein one or more —CH₂— group(s) of the alkyl, alkenyl and alkynyl radicals is/are optionally replaced with —O—, —S—, —SO—, —SO₂— and/or —NR₅—, and the alkyl, alkenyl and alkynyl radicals are unsubstituted or substituted with one or more substituent(s) chosen from an oxo group and a hydroxyl group;

R₅ and R₆ are independently chosen from a lone pair of electrons, a hydrogen radical, C₁-C₈ alkyl radicals, C₂-C₈ alkenyl radicals and C₂-C₈ alkynyl radicals, wherein the alkyl, alkenyl and alkynyl radicals are unsubstituted or substituted with a substituent chosen from a phenyl radical and substituted phenyl radicals;

R₁ is chosen from a hydrogen radical, C₁-C₈ alkyl radicals, C₃-C₈ cycloalkyl radicals, C₂-C₈ alkenyl radicals, C₃-C₈ cycloalkenyl radicals, C₂-C₈ alkynyl radicals and C₃-C₈ cycloalkynyl radicals;

L is chosen from a direct bond, C₁-C₁₂ alkylene radicals, C₂-C₁₂ alkenylene radicals and C₂-C₁₂ alkynylene radicals, wherein one or more —CH₂-group(s) of the alkylene, alkenylene and alkynylene radicals is/are optionally replaced with —O—, —S—, —SO₂—, —NR₅—, C₃-C₈ cycloalkylene and/or C₃-C₈ heterocycloalkylene, and the alkylene, alkenylene and alkynylene radicals are unsubstituted or substituted with one or more substituent(s) independently chosen from an oxo group and a hydroxyl group; and

X is chosen from moieties of formula A, B, C, D, E, F, G, H, I, J, K, L, M, N, O, P, Q, R, S, T, U, V, W or Y 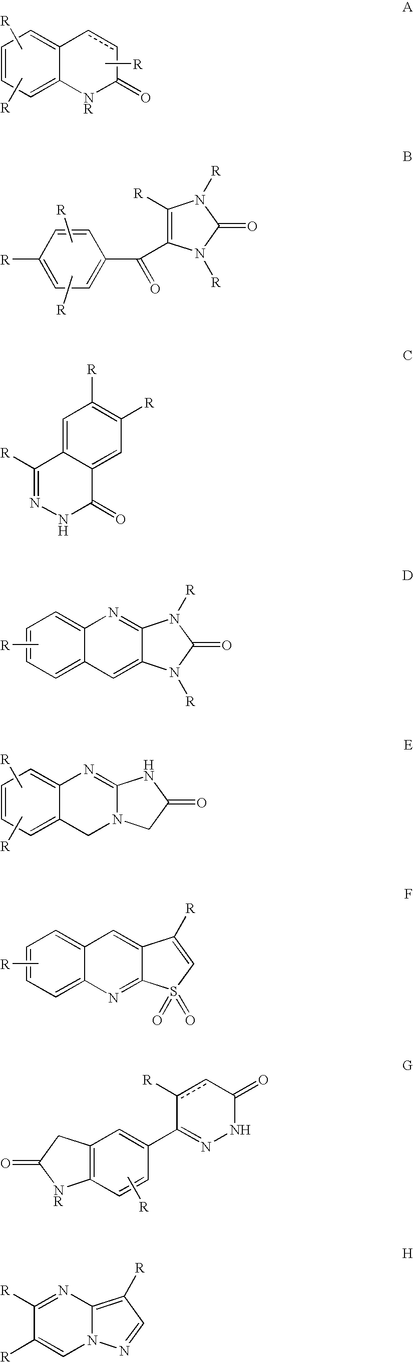 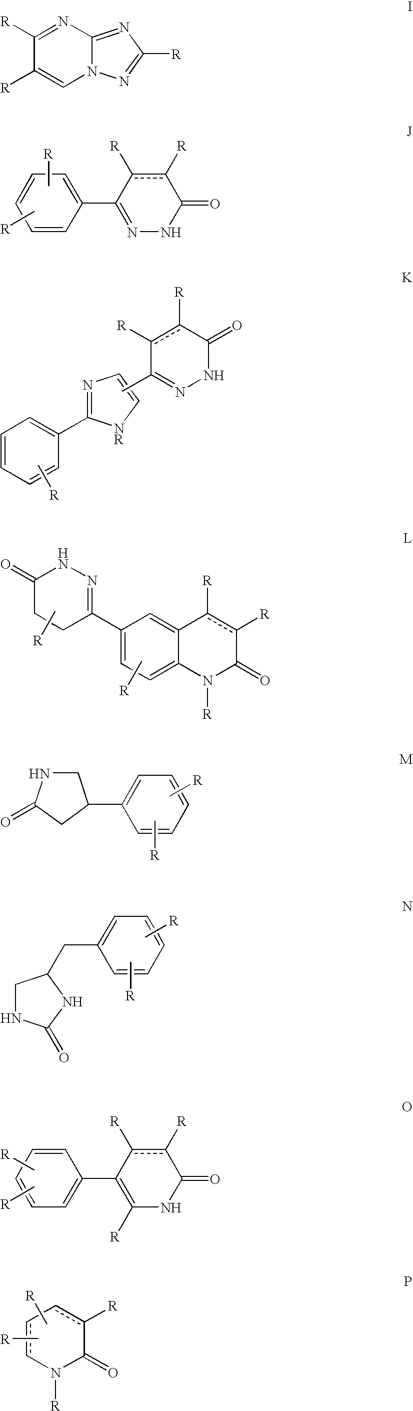 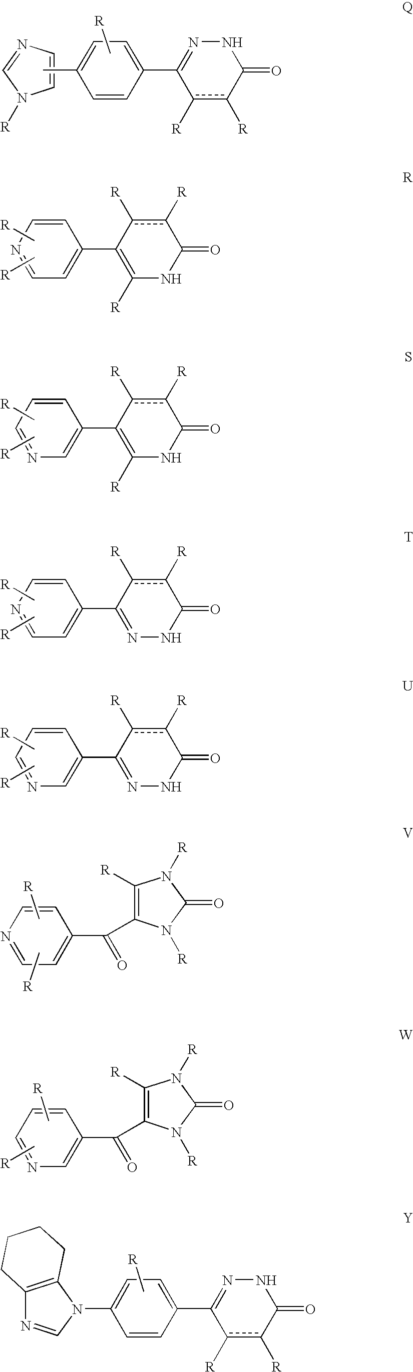
with X connected to L through any one R; and

wherein one R group of moieties A-Y forms a covalent bond between X and L when m is 1, or between X and Ar when n is 1 and m is 0, or between X and β when n is 0 and m is 0; and each remaining R group of moieties A-Y is independently chosen from a hydrogen radical, halo radicals, a nitro group, a cyano group, a trifluoromethyl group, an amino group, NR₅R₆ groups, C₁-C₄ alkoxy radicals, C₁-C₄ alkylthio radicals, COOR₁ radicals, C₁-C₁₂ alkyl radicals, C₂-C₁₂ alkenyl radicals and C₂-C₁₂ alkynyl radicals, wherein one or more —CH₂-group(s) of the alkyl, alkenyl and alkynyl radicals is/are optionally replaced with —O—, —S—, —SO₂— and/or —NR₅—, and the alkyl, alkenyl and alkynyl radicals are unsubstituted or substituted with one or more substituent(s) chosen from an oxo group and a hydroxyl group.

Every variable substituent is defined independently at each occurrence. Thus, the definition of a variable substituent in one part of a formula is independent of its definition(s) elsewhere in that formula and of its definition(s) in other formulas.

In formula (I), moieties A, G, J-L, O-U and Y contain dashed lines in their respective structures. These dashed lines indicate that saturation is optional.

In some embodiments, formula (I)'s Ar is chosen from groups Ar₁, Ar₂, Ar₃, Ar₄, Ar₅, Ar₆ and Ar₇: 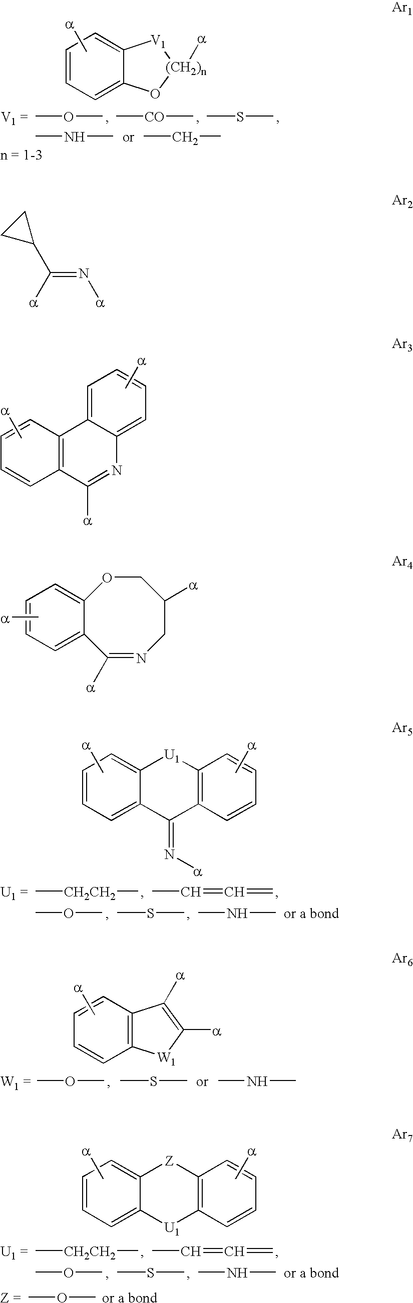
wherein α indicates the position where Ar may bond to β, L, and X. In some embodiments, when X is chosen from moieties of formulas A, B, C, D, E, F, G, H, I, J, K, L, M, N, O, P, Q, R, S, T, U, V, W, and Y, then Ar is group Ar₇, wherein Z is a bond.

In some embodiments, formula (I)'s Ar is a phenyl radical. In further embodiments, the phenyl radical is unsubstituted.

In some embodiments, formula (I)'s Ar is chosen group Ar₇. In further embodiments, group Ar₇'s Z is a bond. In yet further embodiments, group Ar₇'s U₁ is —NH—.

In formula (I)'s β, the N-substituted-2-amino-1-hydroxyeth-1-yl radicals, the N—N-disubstituted-2-amino-1-hydroxyeth-1-yl radicals, the N-substituted-3-amino-2-hydroxypropoxy radicals, and N—N-disubstituted-3-amino-2-hydroxypropoxy radicals may be substituted with any group capable of bonding to such radicals.

In some embodiments, formula (I)'s β is chosen from radicals of formula (β₁) and radicals of formula (β₂):
—CHOHCH₂NZ₁Z₂ (β₁); and
—OCH₂CHOHCH₂NZ₁Z₂ (β₂);
wherein Z₁ and Z₂ are independently chosen from a hydrogen radical, R₁ radicals, and —CH₂CH₂—Y₁—R₁ radicals; and wherein Y₁ is chosen from a —NHCO— radical, a —NHCONH— radical, and a —NHSO₂— radical.

In further embodiments, formula (I)'s β is —OCH₂CHOHCH₂NZ₁Z₂. In yet further embodiments, formula (I)'s Z₁ and Z₂ are independently selected from a hydrogen radical and R₁ radicals. In even further embodiments, Z₁ is hydrogen and Z₂ is C₁-C₄ alkyl. In even further embodiments, Z₂ is isopropyl or tert-butyl.

In some embodiments, formula (I)'s L is chosen from C₁-C₁₂ alkylene radicals, wherein one or more —CH₂— group(s) of the alkylene radicals is/are replaced with —O— and/or —NR₅—, and/or the alkylene radicals are substituted with one or more oxo group(s). In further embodiments, L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4.

In some embodiments, L is —(CH₂)ₚO(CH₂)_{q}O—, wherein q is 1, 2, 3 or 4. In further embodiments, p is 0 or 1. In yet further embodiments, L is —O(CH₂)₃O— or —CH₂O(CH₂)₃O—.

In some embodiments, L is —(CH₂)ₚO—, wherein p is 1, 2, 3 or 4. In further embodiments, L is —(CH₂)₂O—.

In some embodiments, L is —(CH₂)ₚNH(CO)(CH₂)_{q}O—, wherein p and q are independently 1, 2, 3 or 4. In further embodiments, p is 0 or 1. In yet further embodiments, L is —CH₂NH(CO)CH₂O— or —(CH₂)₂NH(CO)CH₂O—.

In some embodiments, L is —(CH₂)ₚ(CO)NH(CH₂)_{q}NH(CO)(CH₂)ᵣO—, wherein q and r are independently 1, 2, 3 or 4. In further embodiments, p is 0 or 1. In yet further embodiments, L is —(CO)NH(CH₂)₂NH(CO)CH₂O—, —CH₂(CO)NH(CH₂)₂NH(CO)CH₂O—, or —(CH₂)₂(CO)NH(CH₂)₂NH(CO)CH₂O—.

In some embodiments, L is chosen from C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are replaced with —C₃-C₈ cycloalkylene and/or C₃-C₈ heterocycloalkylene.

In some embodiments, formula (I)'s X is chosen from moieties of formulas R, S and T, U, V, W and Y. In other embodiments, formula (I)'s X is chosen from moieties of formula S. In yet other embodiments, formula (I)'s X is chosen from moieties of formula J.

In some embodiments, formula (I)'s R groups of moieties A-Y are independently chosen from a hydrogen radical; C₁-C₁₂ alkyl radicals; C₂-C₁₂ alkenyl radicals; C₂-C₁₂ alkynyl radicals, halo radicals and cyano group. In further embodiments, formula (I)'s R groups of moieties A-Y are independently chosen from a hydrogen radical and halo radicals. In yet further embodiments, formula (I)'s R groups of moieties A-Y are independently chosen from a hydrogen radical and a chloro radical.

In some embodiments, formula (I)'s R₁ is chosen from a hydrogen radical, C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

In some embodiments, formula (I)'s R₂, R₃ and R₄ are independently chosen from a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals. In some embodiments, the acylaminoalkyl radicals contain an alkyl chain having from C₁-C₆.

In some embodiments, formula (I)'s R₅ and R₆ are independently chosen from a lone pair of electrons; a hydrogen radical; C₁-C₈ alkyl radicals; C₂-C₈ alkenyl radicals; and C₂-C₈ alkynyl radicals.

Since the compounds of the present invention may possess one or more asymmetric carbon center(s), they may be capable of existing in the form of optical isomers as well as in the form of racemic or non-racemic mixtures of optical isomers. The optical isomers can be obtained by resolution of the racemic mixtures according to conventional processes. One such process entails formation of diastereoisomeric salts, by treatment with an optically active acid or base, and then separation of the mixture of diastereoisomers by crystallization, followed by liberation of the optically active bases from these salts. Examples of appropriate acids are tartaric, diacetyltartaric, dibenzoyltartaric, ditoluoyltartaric, and camphorsulfonic acid.

A different process for separating optical isomers involves the use of a chiral chromatography column optimally chosen to maximize the separation of the enantiomers. Still another available method involves synthesis of covalent diastereoisomeric molecules, for example, esters, amides, acetals, and ketals, by reacting the compounds of the present invention with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the “parent” optically active drug is not necessary prior to dosing the patient, since the compound can behave as a prodrug. The optically active compounds of the present invention likewise can be obtained by utilizing optically active starting materials.

It is understood that the compounds of the present invention encompass individual optical isomers as well as racemic and non-racemic mixtures. In some non-racemic mixtures, the R configuration may be enriched while in other non-racemic mixtures, the S configuration may be enriched.

Accordingly, in some embodiments, formula (I)'s β is chosen from a 2-amino-1-hydroxyeth-1-yl radical, N-substituted-2-amino-1-hydroxyeth-1-yl radicals, and N—N-disubstituted-2-amino-1-hydroxyeth-1-yl radicals, wherein the carbon at position 1 of each radical is enriched over its mirror image counterpart. In some embodiments, the R configuration is enriched.

In some embodiments, formula (I)'s β is chosen from a 3-amino-2-hydroxypropoxy radical, N-substituted-3-amino-2-hydroxypropoxy radicals, and N—N-disubstituted-3-amino-2-hydroxypropoxy radicals, wherein the carbon at position 2 of each radical is enriched over its mirror image counterpart. In some embodiments, the S configuration is enriched.

In some embodiments, formula (I)'s β is chosen from radicals of formula (β₁*) and radicals of formula (β₂*):
—C*HOHCH₂NZ₁Z₂ (β₁*); and
—OCH₂C*HOHCH₂NZ₁Z₂ (β₂*);
wherein the * on the C's in β₁* and β₂* denote chiral centers that are enriched over their respective mirror image counterparts. In some embodiments, formula (I)'s * on the C in β₁* denotes a chiral-carbon center that is enriched in the R configuration. In some embodiments, formula (I)'s * on the C in β₂* denotes a chiral-carbon center that is enriched in the S configuration.

In some embodiments, m+n is 0. In other embodiments, m+n is 1. In other embodiments, m+n is 2.

In some embodiments, the compound of the present invention is chosen from pharmaceutically acceptable salts of compounds of formula (I).

In some embodiments, the compound of the present invention is chosen from hydrates of compounds of formula (I).

In some embodiments, the compound of the present invention is chosen from solvates of compounds of formula (I).

In some embodiments, the compound of the present invention is chosen from metabolites of compounds of formula (I).

In some embodiments, the compound of the present invention is chosen from prodrugs of compounds of formula (I).

In some embodiments, the compound of the present invention is chosen from isosteres of compounds of formula (I).

In some embodiments, the compound of the present invention is chosen from those of formula (I) as defined above, pharmaceutically acceptable equivalents and stereoisomers thereof, wherein:

m is 1;

n is 1;

β is —OCH₂CHOHCH₂NZ₁Z₂;

Ar is phenyl;

L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4; and

X is chosen from moieties of formula J. In further embodiments, the R groups of the moieties of formula J are independently chosen from a hydrogen radical and halo radicals. In yet further embodiments, X is

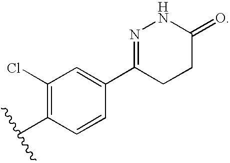

In yet further embodiments, L is chosen from —O(CH₂)₃O—, —CH₂O(CH₂)₃O—, —(CH₂)₂O—, —CH₂NH(CO)CH₂O—, —(CH₂)₂NH(CO)CH₂O—. —(CO)NH(CH₂)₂NH(CO)CH₂O—, —CH₂(CO)NH(CH₂)₂NH(CO)CH₂O—, or —(CH₂)₂(CO)NH(CH₂)₂NH(CO)CH₂O—. In yet further embodiments, Z₁ and Z₂ are independently selected from a hydrogen radical and R₁ radicals. In yet further embodiments, Z₁ is hydrogen and Z₂ is C₁-C₄ alkyl. In yet further embodiments, Z₂ is isopropyl or tert-butyl. In even further embodiments, the compound of the present invention is a non-racemic mixture.

In some embodiments, the compound of the present invention is chosen from those of formula (I) as defined above, pharmaceutically acceptable equivalents and stereoisomers thereof, wherein:

m is 1;

n is 1;

β is as defined above;

Ar is as defined above;

L is as defined above; and

X is as defined above;

provided that when X is chosen from moieties of formulas A, B, C, D, E, F, G, H, I, J, K, L, M, N, O, P and Q, then Ar is group Ar₇, wherein Z is a bond. In further embodiments, β is —OCH₂CHOHCH₂NZ₁Z₂ and X is chosen from moieties of formula J. In yet further embodiments, the R groups of moiety J are independently chosen from a hydrogen radical and halo radicals. In yet further embodiments, L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4. In yet further embodiments, Z₁ and Z₂ are independently selected from a hydrogen radical and R₁ radicals. In yet further embodiments, Z₁ is hydrogen and Z₂ is C₁-C₄ alkyl. In yet further embodiments, Z₂ is isopropyl or tert-butyl. In even further embodiments, the compound of the present invention is a non-racemic mixture.

In some embodiments, the compound of the present invention is chosen from those of formula (I) as defined above, pharmaceutically acceptable equivalents and stereoisomers thereof, wherein:

m is 1;

n is 1;

β is as defined above;

Ar is as defined above;

L is as defined above; and

X is chosen from moieties of formulas R, S, T, U, V, W and Y. In further embodiments, X is chosen from moieties of formula S. In yet further embodiments, β is —OCH₂CHOHCH₂NZ₁Z₂. In yet further embodiments, the R groups of moieties R, S, T, U, V, W and Y are independently chosen from a hydrogen radical and halo radicals, and L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4. In yet further embodiments, Z₁ and Z₂ are independently selected from a hydrogen radical and R₁ radicals. In yet further embodiments, Z₁ is hydrogen and Z₂ is C₁-C₄ alkyl. In yet further embodiments, Z₂ is isopropyl or tert-butyl. In even further embodiments, the compound of the present invention is a non-racemic mixture.

In some embodiments, the compound of the present invention is chosen from those of formula (I) as defined above, pharmaceutically acceptable equivalents and stereoisomers thereof, wherein:

m is 1;

n is 1;

β is as defined above;

Ar is as defined above;

L is as defined above; and

X is as defined above, provided that when X is chosen from moieties of formulas A, B, C, D, E, F, G, H, I, J, K, L, M, N, O, P and Q, then L is chosen from C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are replaced with —C₃-C₈ cycloalkylene and/or C₃-C₈ heterocycloalkylene. In further embodiments, X is chosen from moieties of formula J, R, S, T, U, V, W and Y. In yet further embodiments, β is —OCH₂CHOHCH₂NZ₁Z₂. In yet further embodiments, the R groups moieties R, S, T, U, V, W and Y are independently chosen from a hydrogen radical and halo radicals, and L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4. In yet further embodiments, Z₁ and Z₂ are independently selected from a hydrogen radical and R₁ radicals. In yet further embodiments, Z₁ is hydrogen and Z₂ is C₁-C₄ alkyl. In yet further embodiments, Z₂ is isopropyl or tert-butyl. In even further embodiments, the compound of the present invention is a non-racemic mixture.

Nonlimiting examples of compounds of the present invention include:
N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-acetamide (7);
N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylamino-propoxy)-phenyl]-acetamide (12b);
N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-benzamide (12a);
4-((S)-3-tert-Butylamino-2-hydroxy-propoxy)-N-(2-{2-[2-chloro-4-(6-oxo-1,4,5,6-tetrahydropyridazin-3-yl)-phenoxy]-acetylamino}-ethyl)-benzamide (13);
2-[2-chloro-4-(6-oxo-1,4,5,6-tetrahydro-pyridazin-3-yl)-phenoxy]-N-[4-(2-hydroxy-3-isopropylamino-propoxy)-benzyl]-acetamide (17a);
N-[4-(3-tert-butylamino-2-hydroxy-propoxy)-benzyl]-2-[2-chloro-4-(6-oxo-1,4,5,6-tetrahydro-pyridazin-3-yl)-phenoxy]-acetamide (17b);
2-[2-chloro-4-(6-oxo-1,4,5,6-tetrahydro-pyridazin-3-yl)-phenoxy]-N-{2-[4-(2-hydroxy-3-isopropylamino-propoxy)-phenyl]-ethyl}-acetamide (17c);
N-{2-[4-(3-tert-butylamino-2-hydroxy-propoxy)-phenyl]-ethyl}-2-[2-chloro-4-(6-oxo-1,4,5,6-tetrahydro-pyridazin-3-yl)-phenoxy]-acetamide (17d);
N-{2-[4-((S)-3-tert-Butylamino-2-hydroxy-propoxy)-phenyl]-ethyl}-2-[4-(6-oxo-1,4,5,6-tetrahydro-pyridazin-3-yl)-phenoxy]-acetamide (21);
6-(3-Chloro-4-{3-[4-(2-hydroxy-3-isopropylamino-propoxy)-benzyloxy]-propoxy}-phenyl)-4,5-dihydro-2H-pyridazin-3-one (31a);
6-(4-{3-[4-(3-tert-Butylamino-2-hydroxy-propoxy)-benzyloxy]-propoxy}-3-chloro-phenyl)-4,5-dihydro-2H-pyridazin-3-one (31b);
6-(3-chloro-4-{2-[4-(2-hydroxy-3-isopropylamino-propoxy)-phenyl]-ethoxy}-phenyl)-4,5-dihydro-2H-pyridazin-3-one (37a);
6-(4-{2-[4-(3-tert-butylamino-2-hydroxy-propoxy)-phenyl]-ethoxy}-3-chloro-phenyl)-4,5-dihydro-2H-pyridazin-3-one (37b);
6-[3-Chloro-4-(3-{2-[4-((S)-2-hydroxy-3-isopropylamino-propoxy)-phenyl]-ethoxy}-propoxy)-phenyl]-4,5-dihydro-2H-pyridazin-3-one (46a);
6-[4-(3-{2-[4-((S)-3-tert-Butylamino-2-hydroxy-propoxy)-phenyl]-ethoxy}-propoxy)-3-chloro-phenyl]-4,5-dihydro-2H-pyridazin-3-one (46b);
2′{3-[4-(2-hydroxy-3-isopropylamino-propoxy)-phenoxy]-propoxy}-2-methyl-6-oxo-1,6-dihydro-[3,4′]bipyridinyl-5-carbonitrile;
6′-{3-[4-2-hydroxy-3-isopropylamino-propoxy)-phenoxy]-propoxy}-2-methyl-6-oxo-1,6-dihydro-[3,3′]bipyridinyl-5-carbonitrile;
6-[3-chloro-4-(2-{[4-(2-hydroxy-3-isopropylamino-propoxy)-9H-carbazol-1-yl]-methyl-amino]-ethoxy)-phenyl]-4,5-dihydro-2H-pyridazin-3-one;
6-[4-(2-{[4-(3-tert-butylamino-2-hydroxy-propoxy)-9H-carbazol-1-yl]-methyl-amino}-ethoxy)-3-chloro-phenyl]-4,5-dihydro-2H-pyridazin-3-one; and
6-(3-chloro-4-{2-[(4-{2-hydroxyl-3-[2-(2-methoxy-phenoxy)-ethylamino]-propoxy}-9H-carbazol-1-yl)-methyl-amino]-ethoxy}-phenyl)-4,5-dihydro-2H-pyridazin-3-one.

In one embodiment, a compound of the present invention has a phosphodiesterase-3 inhibition IC₅₀ value of less than 1 μM, while in other embodiments, a compound of the present invention has a phosphodiesterase-3 inhibition IC₅₀ value of less than 500 nM or less than 100 nM.

In one embodiment, a compound of the present invention has a non-specific beta-adrenergic clockade IC₅₀ value of less than 1 μM, while in other embodiments, a compound of the present invention has a non-specific beta-adrenergic blockade IC₅₀ value of less than 500 nM or less than 100 nM.

### Pharmaceutical Compositions

This invention further provides a pharmaceutical composition comprising a compound of the present invention. In one embodiment, the pharmaceutical composition comprises:

(i) an effective amount of a compound of the present invention; and

(ii) a pharmaceutically-acceptable carrier.

In some embodiments, the pharmaceutically-acceptable carrier is chosen from wetting agents, buffering agents, suspending agents, lubricating agents, emulsifiers, disintegrants, absorbents, preservatives, surfactants, colorants, flavorants, sweeteners, and therapeutic agents other than those compounds of the present invention.

In some embodiments, the pharmaceutically-acceptable carrier is chosen from fillers, diluents, excipients, and solvent encapsulating materials. In some embodiments, the pharmaceutically-acceptable carrier is active with respect to the patient. In some embodiments, the pharmaceutically-acceptable carrier are chosen from: (1) sugars, such as lactose, glucose and sucrose; (2) starches, such as corn starch and potato starch; (3) cellulose band its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; and (21) polyesters, polycarbonates and polyanhydrides.

In some embodiments, the pharmaceutically-acceptable carrier is liquid and in others it is solid.

The inventive pharmaceutical composition may be formulated for administration in solid or liquid form, including those adapted for the following: (1) oral administration, for example, drenches (for example, aqueous or non-aqueous solutions or suspensions), tablets, (for example, those targeted for buccal, sublingual, and systemic absorption), boluses, powders, granules, pastes for application to the tongue, hard gelatin capsules, soft gelatin capsules, mouth sprays, emulsions and microemulsions; (2) parenteral administration, for example, by subcutaneous, intramuscular, intravenous or epidural injection as, for example, a sterile solution or suspension, or a sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

### Methods of Use

The present invention further provides a method for regulating calcium homeostasis, comprising administering an effective amount of a compound of the present invention to an animal in need of such regulation. Animals include both human and non-human animals, including, but not limited to, mammals.

The present invention further provides a method for treating a disease, disorder or condition in which disregulation of calcium homeostasis is implicated, comprising administering an effective amount of a compound of the present invention to an animal in need of such treatment.

The present invention also provides a method for treating cardiovascular disease, stroke, epilepsy, an ophthalmic disorder or migraine, comprising administering an effective amount of a compound of the present invention to an animal in need of such treatment.

In some embodiments of the inventive method, the cardiovascular disease is heart failure, hypertension, SA/AV node disturbance, arrhythmia, hypertrophic subaortic stenosis or angina. In other embodiments of the inventive method, the heart failure is chronic heart failure or congestive heart failure.

The present invention further provides a method of inhibiting β-adrenergic receptors and/or inhibiting phosphodiesterase PDE, including PDE3, comprising administering an effective amount of a compound of the present invention to an animal in need of such treatment.

The compound of the present invention may be administered by any means known to an ordinarily skilled artisan. For example, the compound of the present invention may be administered orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrasternal, intracranial, and intraosseous injection and infusion techniques. The exact administration protocol will vary depending upon various factors including the age, body weight, general health, sex and diet of the patient; the determination of specific administration procedures would be routine.

The compound of the present invention may be administered by a single dose, multiple discrete doses, or continuous infusion. Pump means, particularly subcutaneous pump means, are useful for continuous infusion.

Dose levels on the order of about 0.001 mg/kg/d to about 10,000 mg/kg/d of compound of the present invention are useful for the inventive method, with preferred levels being about 0.1 mg/kg/d to about 1,000 mg/kg/d, and more preferred levels being about 1 mg/kg/d to about 100 mg/kg/d. The specific dose level for any particular patient will vary depending upon a variety of factors, including the activity and the possible toxicity of the specific compound employed; the age, body weight, general health, sex, and diet of the patient; the time of administration; the rate of excretion; drug combination; the severity of the congestive heart failure, and the form of administration. Typically, in vitro dosage-effect results provide useful guidance on the proper doses for patient administration. Studies in animal models are also helpful. The considerations for determining the proper dose levels are well known in the art and within the skill of a physician.

Any administration regimen well known to an ordinarily skilled artisan for regulating the timing and sequence of drug delivery can be used and repeated as necessary to effect treatment in the inventive method. A further regimen may include pretreatment and/or co-administration with additional therapeutic agents.

The compound of the present invention can be administered alone or in combination with one or more additional therapeutic agent(s) for simultaneous, separate, or sequential use. The additional agent(s) can be any therapeutic agent(s), including without limitation one or more compound(s) of the present invention. The compound of the present invention can be co-administered with one or more therapeutic agent(s) either (i) together in a single formulation, or (ii) separately in individual formulations designed for optimal release rates of their respective active agent.

The compounds of the present invention may be readily made. For example, when m+n is 0 and β and X are directly bonded, the compounds of the present invention may be prepared using standard aromatic chemistry known to those skilled in the art. As shown in general Scheme 1 below, protected aryl hydroxyl precursors of moieties X (P₁ may be e.g., acetyl, benzyl, alkylsilyl, or other appropriate protecting group and Q₁, R₇, S₁, T₁ are chosen to reach a particular moiety X) may be deprotected and then may be reacted with epichlorohydrin to yield epoxide intermediates which may be reacted with amines to yield the final products.

Furthermore, such a scheme could readily be adapted to link Ar to β or to link Ar to L or to link Ar to X.

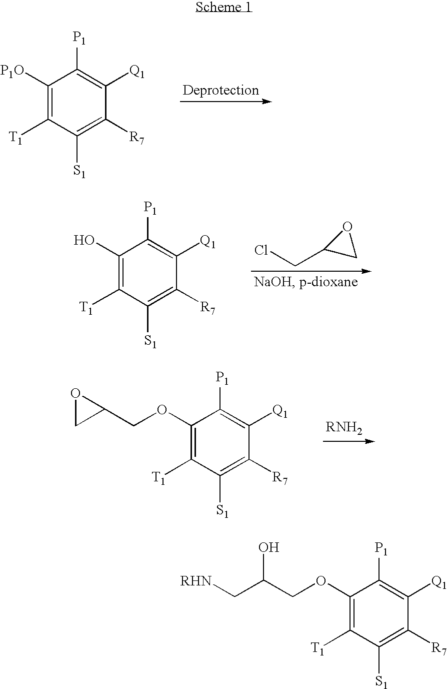

In cases m is 1, wherein X and β or X and Ar are connected by a linker of one or more atoms, the linker may be attached to β, Ar, or X, and the intermediate moiety β-L or X-L or L-Ar may then be linked to X or Ar/β or β/X, respectively, to form β-(Ar)ₙ-L-X.

For example, a general method for preparing β-(Ar)ₙ-L may proceed as follows. Protected phenols of the type depicted below in general Scheme 2 may be reacted with suitably protected linker chains L. “J₂” in the scheme may be any of various species known to those skilled in the art which can be reacted with a hydroxyl group. For example, J₂ may be a bromine atom, which can be displaced by reaction with the anion of the phenol, or J₂ may be an alcohol group which can be reacted with the phenol under Mitsunobu reaction conditions. P₂ may be a suitable protecting group which can be removed under different conditions than those which cleave P₁. The partially deprotected compound may be reacted with a precursor of moiety X or a precursor of Ar, as described in general Scheme 4, before attaching the remaining β constituent. Such a scheme could be readily adapted to link L to Ar or to link β-L to Ar by one of ordinary skill in the art.

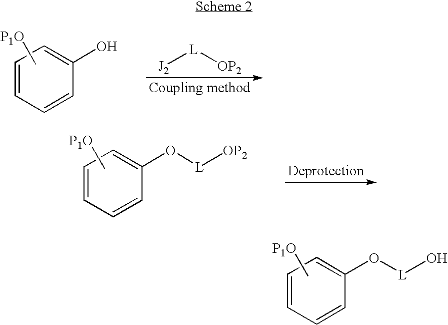

In addition, a general method for preparation of X-(Ar)ₙ-L is analogous to the method for β-(Ar)ₙ-L may proceed as follows. Precursors of moieties X with a hydroxyl group on one of the rings may be reacted with a protected linker group as described in Scheme 2 above and may be subsequently deprotected. Such a scheme could be readily adapted to link X to Ar or to link X to L-(Ar)ₙ-β or to link X to Ar-β by one of ordinary skill in the art.

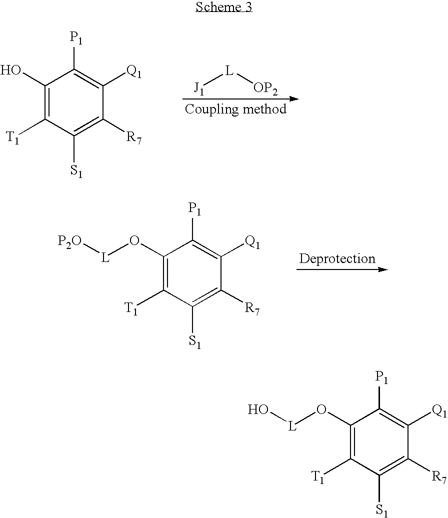

General method for reacting Ar-L or X-L with X or Ar to make Ar-L-X may proceed as follows. A resultant compound from general Scheme 2 may be reacted with an aryl hydroxyl precursor of moiety X via standard Mitsunobu chemistry as shown below in Scheme 4. Following deprotection of the remaining hydroxyl group, sequential reaction with epichlorohydrin and a substituted amine may deliver the final product.

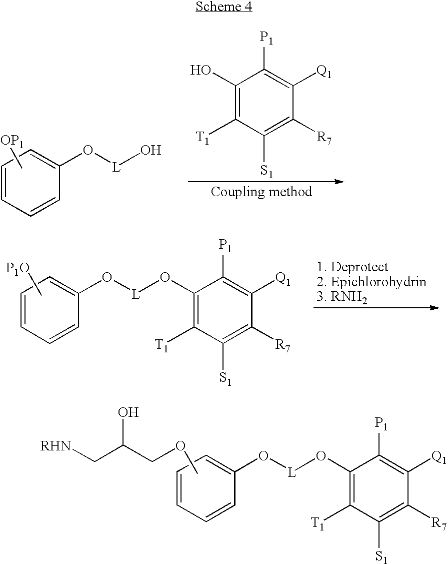

Indeed, general Schemes 1-4 could be readily adapted to make X-(L)ₘ-(Ar)ₙ-β by one of ordinary skill in the art.

A compound from general Scheme 3 may similarly be reacted with a protected phenol, as shown below in Scheme 5, and the coupling product may be converted to the final compound by the same deprotection/reaction with epichlorohydrin/reaction with RNH₂ sequence as previously described.

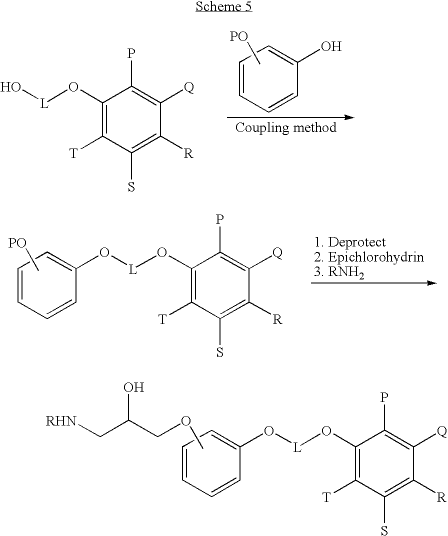

### EXAMPLES

### Example 1

### Synthesis of N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-acetamide (7)

N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-acetamide (7) was synthesized according to Scheme I.

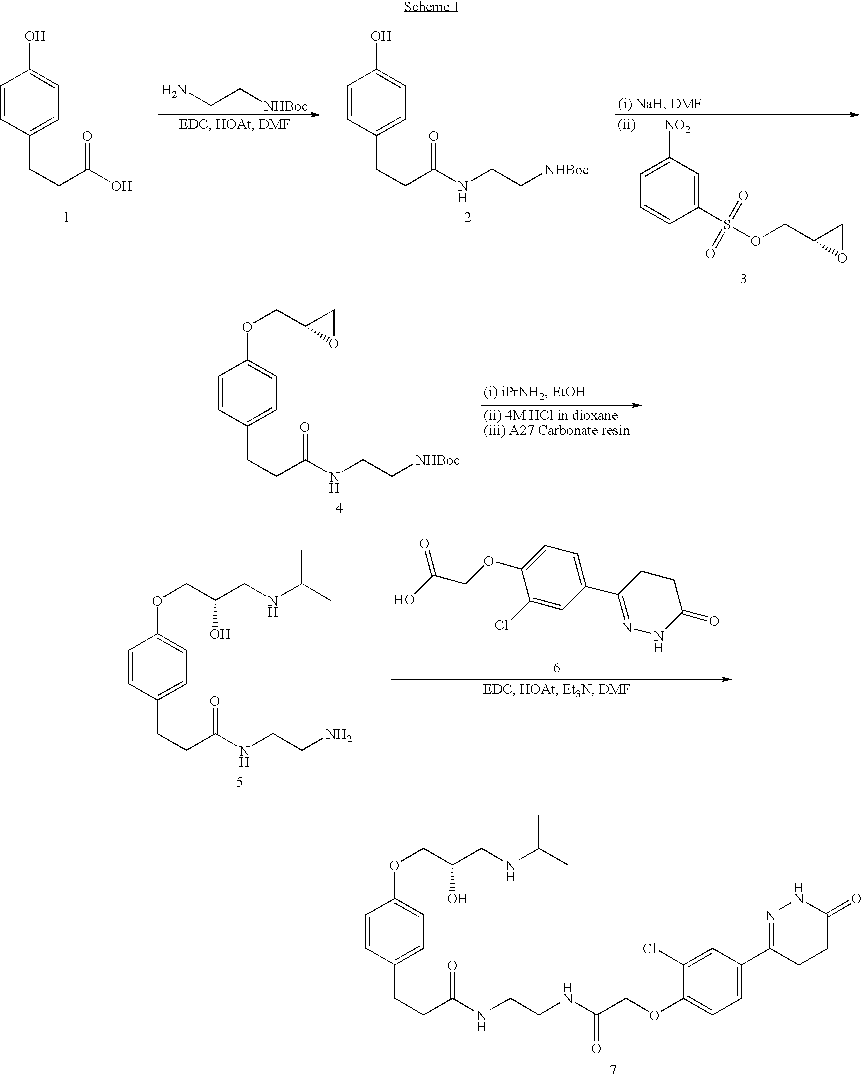

### Synthesis of {2-[2-(4-Hydroxy-phenyl)-propionylamino]-ethyl}-carbamic acid tert butyl ester (2)

To a round bottom flask containing 4-hydroxyphenyl propanoic acid (1) (1.66 g, 10 mmol), (3-dimethylamino-propyl)-ethyl-carbodiimide hydrochloride (EDC.HCl, 2.15 g, 11 mmol), [1,2,3]triazolo[4,5-b]pyridin-3-ol (HOAt, 0.556 g, 4 mmol) and N-(tertbutyloxycarbonyl)ethylene diamine (1.76 g, 11 mmol) was added N,N-dimethylformamide (10 mL). The mixture was stirred at ambient temperature for 18 h then poured onto 50% saturated NH₄Cl (aq.) (60 ml). The mixture was then extracted with ethyl acetate (4×20 ml) and the organic extracts were combined and washed with 1M aqueous citric acid solution (2×40 ml), 1M NaHCO₃ (40 ml), water (2×40 ml) and saturated brine (50 ml). The solution was then dried (Na₂SO₄) and concentrated under reduced pressure to give a colorless foam (2.79 g, 90% yield), 97% pure by LC-MS and >90% pure by ¹H-nmr.

### Synthesis of {2-[3-(4-(S)-Oxiranylmethoxy-phenyl)-propionylamino]-ethyl}-carbamic acid tert butyl ester (4)

To a stirred solution of {2-[2-(4-hydroxy-phenyl)-propionylamino]-ethyl}-carbamic acid tert butyl ester (2) (1.22 g, 4.33 mmol) in N,N-dimethylformamide (8 mL) was added sodium hydride (60% dispersion in mineral oil) (182 mg, 4.55 mmol) and the mixture was stirred at ambient temperature for 20 minutes. Following this, (2S)-3-nitro-benzenesulfonic acid oxiranylmethyl ester (3, 1.20 g, 4.55 mmol) was added and the reaction mixture was stirred at ambient temperature for 18 hours. Following this, 50% saturated NH₄Cl (aq.) (100 ml) was added then the mixture was then extracted with ethyl acetate (4×20 ml) and the organic extracts were washed with 1N NaOH (aq.) (30 ml), 50% aq. saturated brine (3×30 ml) and aq. saturated brine (50 ml). The combined organic extracts were then dried (Na₂SO₄) and concentrated under reduced pressure to leave a colorless solid as the crude product. This solid was purified by flash column chromatography over silica gel (gradient eluent=0-65% ethyl acetate in dichloromethane) to afford a pale yellow solid (1.25 g, 85% yield) of purity>95% by LCMS and ¹H NMR.

### Synthesis of N-(2-Amino-ethyl)-3-[4-(2-(S)-hydroxy-3-isopropylamino-propoxy)-phenyl]-propionamide (5)

{2-[3-(4-(S)-Oxiranylmethoxy-phenyl)-propionylamino]-ethyl}-carbamic acid tert butyl ester (4) (1.25 g, 3.4 mmol) was suspended in ethanol (35 ml) at ambient temperature and isopropylamine (3 ml, 34 mmol) was added. The reaction mixture was heated to reflux and stirred at this temperature for 2 h. Following this, the reaction mixture was allowed to cool and concentrated under reduced pressure. The residue was then dissolved in methanol (5 ml) and the solution was cooled to 0° C. then to this was added 4M HCl in dioxane (35 ml, 136 mmol) and the reaction mixture was stirred at 0° C. for 2 h. The solution was then concentrated under reduced pressure then dissolved in methanol (35 ml). A27 carbonate resin (23.1 g, 35 mmol) was then added and the suspension was stirred at ambient temperature for 1 h before being filtered and the filtrate was concentrated under reduced pressure to afford a colorless solid (848 mg, 84% yield) of purity>90% by ¹H NMR.

### Synthesis of N-(2-{2-[2-Chloro-4-(6-oxo-1,4,5,6-tetrahydro-pyridazin-3-yl)-phenoxy]-acetylamino}-ethyl)-2-(4-((S)-2-hydroxy-3-isopropylaminopropoxy)-phenyl]-propionamide (7)

To a round bottom flask was added (3-dimethylamino-propyl)-ethyl-carbodiimide hydrochloride (EDC-HCl, 0.273 g, 1.40 mmol), [1,2,3]triazolo[4,5-b]pyridin-3-ol (HOAt, 0.119 g, 0.86 mmol), [2-chloro-4-(6-oxo-1,4,5,6-tetrahydro-pyridazin-3-yl)-phenoxy]-acetic acid (6, 0.198 g, 0.7 mmol) and N,N-dimethylformamide (2 mL) and the solution was stirred at ambient temperature for 1 h. A suspension of N-(2-amino-ethyl)-3-[4-(2-(S)-hydroxy-3-isopropylamino-propoxy)-phenyl]-propionamide (5) (0.28 g, 0.78 mmol) in N,N-dimethylformamide (2 mL) was then added and the reaction mixture was stirred at ambient temperature for 18 h. Following this, H₂O (40 ml) was added and the pH was adjusted to pH 11 using 2N NaOH (aq.). The mixture was then extracted with ethyl acetate (4×20 ml) and the organic extracts were combined, washed with 50% saturated brine (4×30 ml) and brine (40 ml) then dried (Na₂SO₄) and concentrated under reduced pressure to afford the crude product. This product was purified by preparative HPLC to afford an off-white powder (28 mg, 6% yield) which was 100% pure by 10 min. LCMS (UV@215 nm: retention time=3.27 min., peak area=100%, TOF-ES⁺ with 25 eV cone voltage: m/z=588.3 (100%) & 590.3 (45%)). ¹H NMR: ([D₄]-MeOH, δ in ppm): 7.79 (1H, d, J=2.8 Hz), 7.57 (1H, dd, J¹=8.8 Hz, J²=2.4 Hz), 7.01-6.93 (4H, m), 6.73 (2H, m), 4.52 (2H, s), 3.92 (1H, m), 3.81 (1H, s), 3.80 (1H, d, J=1.6 Hz), 3.25 (2H, m), 3.21 (2H, m), 2.82 (2H, t, J=8.0 Hz), 2.78-2.72 (2H, m), 2.68 (2H, t, J=8.0 Hz), 2.56 (1H, m), 2.41 (2H, t, J=8.8 Hz), 2.28 (2H, t, J=8.4 Hz), 1.00 (6H, m).

### Example 2

### Synthesis of N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-acetamide (12b)

N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylamino-propoxy)-phenyl]-acetamide (12b) was synthesized according to Scheme II.

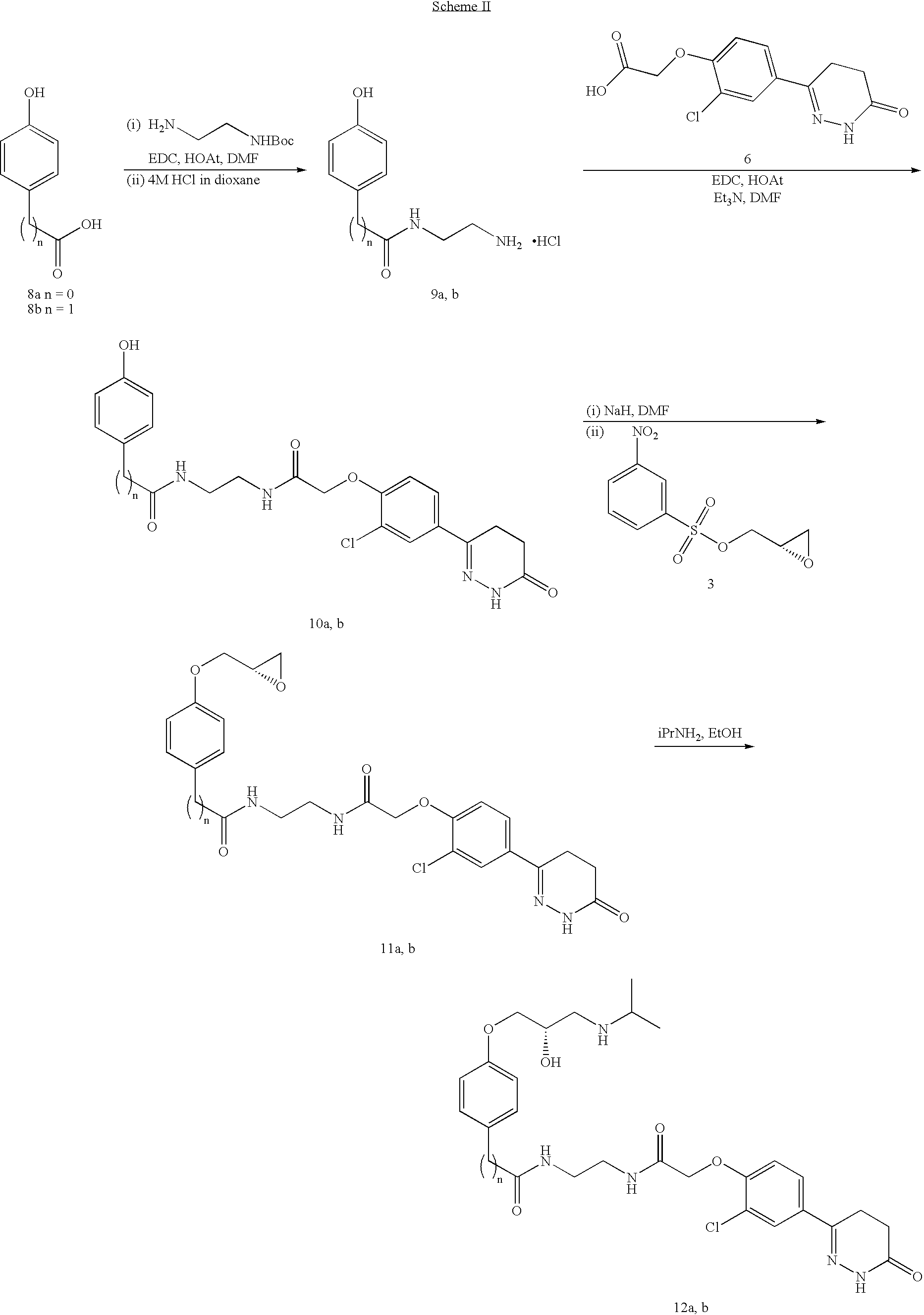

### Synthesis of N-(2-Amino-ethyl)-2-(4-hydroxyphenyl)-acetamide hydrochloride (9b)

The first stage of this synthesis was carried out according to the procedure for (2) above except 4-hydroxyphenylacetic acid (8b) was used instead of 4-hydroxyphenyl propanoic acid (1). The crude product from this coupling stage was obtained as a colorless solid (2.45 g, 58% corrected yield) of purity 70% by LCMS. This product (1.16 g, 3.93 mmol) was then dissolved in 4M HCl in dioxane (20 ml, 79 mmol) at 0° C. and the reaction mixture was stirred at this temperature for 2 h before being concentrated under reduced pressure. The residue was treated with diethyl ether and the resulting solid was filtered and dried with suction to afford the HCl salt (taken directly to the next stage).

### Synthesis of N-(2-{2-[2-Chloro-4-(6-oxo-1,4,5,6-tetrahydropyridazin-3-yl)-phenoxy]-acetylamino}-ethyl)-2-(4-hydroxy-phenyl)-acetamide (10b)

To a round bottom flask was added (3-dimethylamino-propyl)-ethyl-carbodiimide hydrochloride (EDC-HCl, 0.891 g, 4.3 mmol), [1,2,3]triazolo[4,5-b]pyridin-3-ol (HOAt, 0.621 g, 4.3 mmol), [2-chloro-4-(6-oxo-1,4,5,6-tetrahydro-pyridazin-3-yl)-phenoxy]-acetic acid (6, 1.17 g, 3.93 mmol) and N,N-dimethylformamide (10 mL). This mixture was then stirred at ambient temperature under nitrogen for 1 h before a solution of N-(2-amino-ethyl)-2-(4-hydroxyphenyl)-acetamide hydrochloride (9b) (0.907 g, 3.93 mmol) and triethylamine (6 ml, 39.3 mmol) in N,N-dimethylformamide (10 mL) was added. The mixture was then stirred at ambient temperature for 18 h then 0.5 M HCl (120 ml) was added. The mixture was then extracted with ethyl acetate (4×30 ml) and the organic extracts were combined, dried (Na₂SO₄) and concentrated to give a precipitate which was filtered. The solid collected was recrystallised from ethanol to afford a colorless solid (0.928 g, 51% yield) which was 98% pure by LCMS (UV).

### Synthesis of N-(2-[2-[2-Chloro-4-(6-oxo-1,4,5,6-tetrahydropyridazin-3-yl)-phenoxy]-acetylamino]-ethyl)-2-[4-((S)-1-oxiranylmethoxy)-phenyl]-acetamide (11b)

This was synthesized in an analogous fashion to (4) above except using N-(2-{2-[2-chloro-4-(6-oxo-1,4,5,6-tetrahydropyridazin-3-yl)-phenoxy]-acetylamino}-ethyl)-2-(4-hydroxy-phenyl)-acetamide (10b) as the starting material. The product was obtained as a colorless solid (0.392 g, 42% yield) of purity>95% by LCMS and ¹H NMR.

### Synthesis of N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylamino-propoxy)-phenyl]-acetamide (12b)

N-(2-{2-[2-Chloro-4-(6-oxo-1,4,5,6-tetrahydropyridazin-3-yl)-phenoxy]-acetylamino}-ethyl)-2-[4-((S)-1-oxiranylmethoxy)-phenyl]-acetamide (11b) (200 mg, 0.39 mmol) was suspended in ethanol (10 ml) at ambient temperature and isopropylamine (1 ml, 11.6 mmol) was added. The reaction mixture was heated to reflux and stirred at this temperature for 7 h. Following this, the reaction mixture was allowed to cool and concentrated under reduced pressure. The residue was then recrystallised from ethanol to afford 12b as a colorless foam (130 mg, 58% yield), 96% pure by LC-MS and ¹H-nmr). 10 min LC-MS (UV@215 nm: retention time=3.28 min., peak area=96%, TOF-ES⁺ with 25 eV cone voltage: m/z=574.28 (100%) & 576.29 (40%)). ¹H NMR: ([D₆]-DMSO, δ in ppm): 10.86 (1H, s), 7.96 (2H, m), 7.85 (1H, d, J=2.4 Hz), 7.69 (1H, dd, J¹=2.0 Hz, J²=8.8 Hz), 7.19 (2H, d, J=8.4 Hz), 7.13 (1H, d, J=8.8 Hz), 6.89 (2H, m), 4.87 (1H, br s), 4.67 (2H, s), 4.07-3.83 (4H, m), 3.36 (2H, s), 3.27-3.17 (4H, m), 2.96 (2H, t, J=8.4 Hz), 2.81-2.69 (2H, m), 2.64-2.55 (2H, m), 2.48 (2H, t, J=8.4 Hz), 1.04 (3H, s), 1.02 (3H, s).

### Synthesis of N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-benzamide (12a)

This was synthesized using the same procedure as 12b above but using 4-hydroxybenzoic acid (8a) in the first step instead of 8b. 12a was obtained as a colorless foam (210 mg, 37% yield, 100% pure by LC-MS and ¹H-nmr). 10 min LC-MS (UV@215 nm: retention time=3.10 min., peak area=100%, TOF-ES⁺ with 25 eV cone voltage: m/z=560.43 (100%) & 562.40 (40%)). ¹H NMR: ([D₆]-DMSO, δ in ppm): 10.98 (1H, s), 8.42 (1H, t, J=5.2 Hz), 8.20 (1H, t, J=5.2 Hz), 7.84 (3H, m), 7.63 (1H, dd, J¹=8.8 Hz, J²=2.4 Hz), 7.10 (1H, d, J=8.8 Hz), 7.04 (2H, m), 5.09 (1H, br s), 4.72 (2H, s), 4.07 (1H, dd, J¹=10.0 Hz, J²=4.8 Hz), 3.97 (1H, dd, J¹=10.0 Hz, J²=4.8 Hz), 3.91 (1H, m), 2.94 (2H, t, J=8.4 Hz), 2.82-2.70 (2H, m), 2.66-2.57 (2H, m), 2.48 (2H, t, J=8.4 Hz), 1.05 (3H, d, J=1.6 Hz), 1.03 (3H, d, J=1.6 Hz).

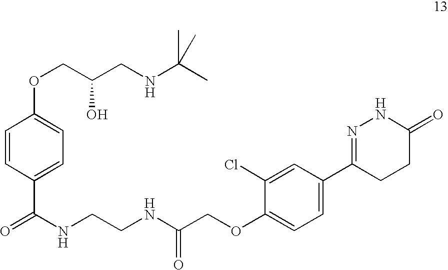

### Synthesis of 4-((S)-3-tert-Butylamino-2-hydroxy-propoxy)-N-(2-{2-[2-chloro-4-(6-oxo-1,4,5,6-tetrahydropyridazin-3-yl)-phenoxy]-acetylamino}-ethyl)-benzamide (13)

This was synthesized using the same procedure as 12a above but using tert-butylamine in the final step instead of iso-propylamine. 13 was obtained as a colorless foam (190 mg, 41% yield over two steps, 100% pure by LC-MS and 1H-nmr). 10 min LC-MS (UV@215 nm: retention time=3.25 min., peak area=100%, TOF-ES+ with 25 eV cone voltage: m/z=574.56 (100%) & 576.52 (40%)). 1H NMR: ([D6]-DMSO, δ in ppm): 10.72 (1H, s), 8.16 (1H, m), 7.94 (1H, m), 7.58 (3H, m), 7.38 (1H, dd, J1=8.8 Hz, J2=2.4 Hz), 6.85 (1H, d, J=8.8 Hz), 6.78 (2H, m), 4.80 (1H, br s), 4.47 (2H, s), 3.84 (1H, dd, J1=10.0 Hz, J2=4.4 Hz), 3.73 (1H, dd, J1=10.0 Hz, J2=6.8 Hz), 3.60 (1H, m), 2.69 (2H, t, J=8.8 Hz), 2.51-2.32 (2H, m), 2.23 (2H, t, J=8.8 Hz), 0.83 (9H, s).

### Example 3

### Synthesis of 2-[2-Chloro-4-(6-oxo-1,4,5,6-tetrahydro-pyridazin-3-yl)-phenoxy]-N-[4-(2-hydroxy-3-isopropylamino-propoxy)-benzyl]-acetamide; N-[4-(3-tert-Butylamino-2-hydroxy-propoxy)-benzyl]-2-[2-chloro-4-(6-oxo-1,4,5,6-tetrahydro-pyridazin-3-yl)-phenoxy]-acetamide; 2-[2-Chloro-4-(6-oxo-1,4,5,6-tetrahydro-pyridazin-3-yl)-phenoxy]-N-{2-[4-(2-hydroxy-3-isopropylamino-propoxy)-phenyl]-ethyl}-acetamide; and N-{2-[4-(3-tert-Butylamino-2-hydroxy-propoxy)-phenyl]-ethyl}-2-[2-chloro-4-(6-oxo-1,4,5,6-tetrahydro-pyridazin-3-yl)-phenoxy]-acetamide (17a, 17b, 17c, and 17d)

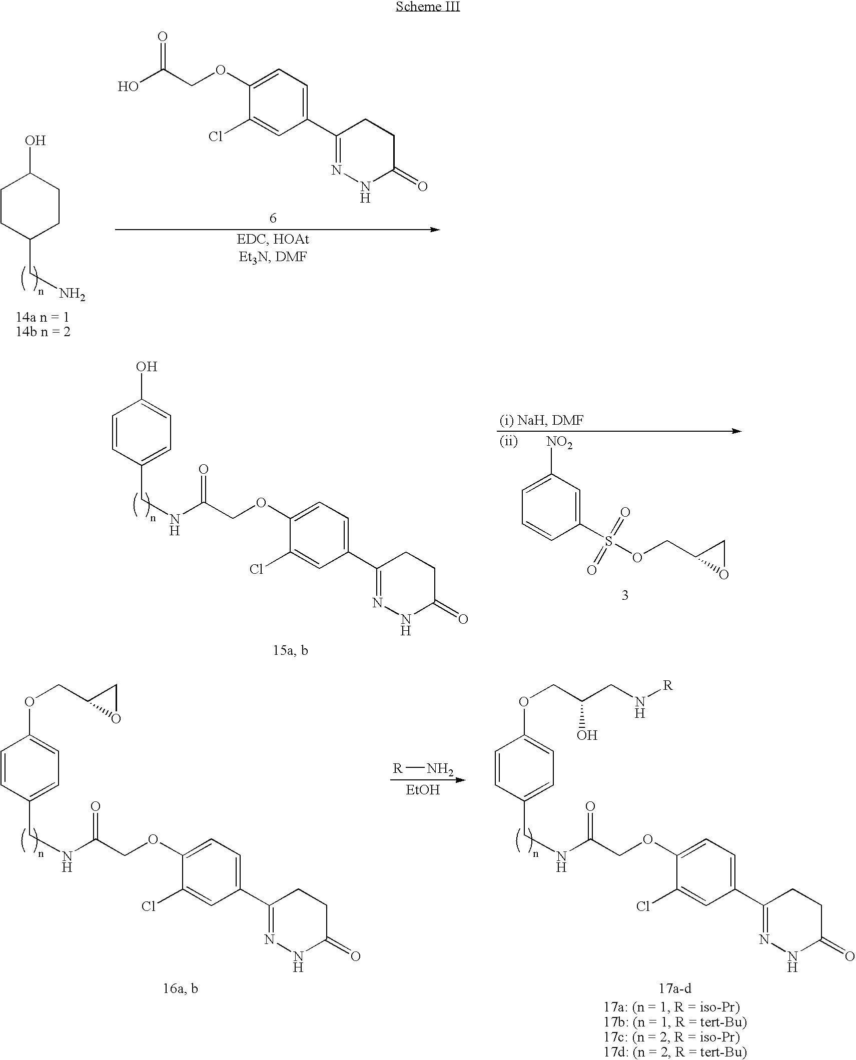

### Synthesis of 2-[2-Chloro-4-(6-oxo-1,4,5,6-tetrahydro-pyridazin-3-yl)-phenoxy]-N-(4-hydroxybenzyl)-acetamide (15a)

To a stirred solution of [2-chloro-4-(6-oxo-1,4,5,6-tetrahydro-pyridazin-3-yl)-phenoxy]-acetic acid (3, 700 mg, 2.48 mmol) in N,N-dimethylformamide (8 mL) was added (3-dimethylamino-propyl)-ethyl-carbodiimide hydrochloride (EDC.HCl, 475 mg, 2.48 mmol) and [1,2,3]triazolo[4,5-b]pyridin-3-ol (HOAt, 337 mg, 2.48 mmol). The mixture was stirred at ambient temperature for 30 minutes until a clear solution and then a solution of 4-aminomethyl-phenol hydrobromide (14a, 505 mg, 2.48 mmol) and trietylamine (415 μL, 2.97 mmol) in N,N-dimethylformamide (4 mL) was added. The mixture was stirred for 5 h at ambient temperature, water (100 mL) was added and the suspension was left standing for 16 h at ambient temperature. The precipitate was filtered off, rinsed with water (2×10 mL) and dried under reduced pressure at 40-50° C. to give 2-[2-chloro-4-(6-oxo-1,4,5,6-tetrahydro-pyridazin-3-yl)-phenoxy]-N-(4-hydroxybenzyl)-acetamide (15a) as light brown powder (537 mg, 56% yield, 92% pure by LC-MS and ¹H-nmr).

### Synthesis of 2-[2-Chloro-4-(6-oxo-1,4,5,6-tetrahydro-pyridazin-3-yl)-phenoxy]-N-[2-(4-hydroxy-phenyl)-ethyl]-acetamide (15b)

To a stirred solution of [2-chloro-4-(6-oxo-1,4,5,6-tetrahydro-pyridazin-3-yl)-phenoxy]-acetic acid (2, 700 mg, 2.48 mmol) in N,N-dimethylformamide (8 mL) was added (3-dimethylamino-propyl)-ethyl-carbodiimide hydrochloride (EDC.HCl, 475 mg, 2.48 mmol) and [1,2,3]triazolo[4,5-b]pyridin-3-ol (HOAt, 337 mg, 2.48 mmol). The mixture was stirred at ambient temperature for 30 minutes until a clear solution and then a solution of 4-(2-amino-ethyl)-phenol (14b, 340 mg, 2.48 mmol) in N,N-dimethylformamide (4 mL) was added. The mixture was stirred for 16 h at ambient temperature and then water (100 mL) was added. The suspension was left standing for 16 h at ambient temperature. The precipitate was filtered off, rinsed with water (2×10 mL) and dried under reduced pressure at 40-50° C. to give 2-[2-chloro-4-(6-oxo-1,4,5,6-tetrahydro-pyridazin-3-yl)-phenoxy]-N-[2-(4-hydroxy-phenyl)-ethyl]-acetamide (15b) as off-white powder (438 mg, 44% yield, >95% pure by LC-MS and ¹H-nmr).

### Synthesis of 2-[2-Chloro-4-(6-oxo-1,4,5,6-tetrahydro-pyridazin-3-yl)-phenoxy]-N-(4-oxiranylmethoxy-benzyl)-acetamide (16a)

To a stirred suspension of sodium hydride (60% dispersion in mineral oil, 61 mg, 1.52 mmol) in N,N-dimethylformamide (1 mL) under N₂ at 0° C. was added a solution of 2-[2-chloro-4-(6-oxo-1,4,5,6-tetrahydro-pyridazin-3-yl)-phenoxy]-N-(4-hydroxybenzyl)-acetamide (15a, 537 mg, 1.38 mmol) in N,N-dimethylformamide (2 mL) and the reaction mixture was stirred at ambient temperature for 20 min. A solution of (2S)-3-nitro-benzenesulfonic acid oxiranylmethyl ester (5, 358 mg, 1.38 mmol) in N,N-dimethylformamide (2 mL) was then added at 0° C. The reaction mixture was stirred at ambient temperature for 16 h, poured onto a mixture of ice-water (15 mL) and saturated aqueous ammonium chloride solution (15 mL) and extracted with ethyl acetate (3×30 mL). The combined organic layers were washed with aqueous 1N sodium hydroxide solution (2×30 mL) and saturated brine (30 ml), dried over sodium sulphate and concentrated under reduced pressure to give 2-[2-chloro-4-(6-oxo-1,4,5,6-tetrahydro-pyridazin-3-yl)-phenoxy]-N-(4-oxiranylmethoxy-benzyl)-acetamide (16a) as a pale yellow solid, which was used for the next reaction step without further purification.

### Synthesis of 2-[2-Chloro-4-(6-oxo-1,4,5,6-tetrahydro-pyridazin-3-yl)-phenoxy]-N-[2-(4-oxiranylmethoxy-phenyl)-ethyl]-acetamide (16b)

16b was synthesized from 15b using the procedure described for 16a. 2-[2-Chloro-4-(6-oxo-1,4,5,6-tetrahydro-pyridazin-3-yl)-phenoxy]-N-[2-(4-oxiranylmethoxy-phenyl)-ethyl]-acetamide (16b) was isolated as pale yellow solid which was used for the next reaction step without further purification.

### Synthesis of 2-[2-Chloro-4-(6-oxo-1,4,5,6-tetrahydro-pyridazin-3-yl)-phenoxy]-N-[4-(2-hydroxy-3-isopropylamino-propoxy)-benzyl]-acetamide (17a)

To a stirred solution of crude 2-[2-chloro-4-(6-oxo-1,4,5,6-tetrahydro-pyridazin-3-yl)-phenoxy]-N-(4-oxiranylmethoxy-benzyl)-acetamide (16a) from the last step in ethanol (20 mL) was added iso-propylamine (562 μL, 7.0 mmol). The mixture was stirred for 3 h under reflux and the solvent was then removed under reduced pressure. The residue was absorbed onto silica (500 mg) from dichloromethane/methanol 5:1, dry-loaded and purified by flash chromatography on silica gel (10 g) eluting with a gradient of 5-10% methanol in dichloromethane to give 2-[2-chloro-4-(6-oxo-1,4,5,6-tetrahydro-pyridazin-3-yl)-phenoxy]-N-[4-(2-hydroxy-3-isopropylamino-propoxy)-benzyl]-acetamide (17a) as colorless solid (162 mg, 23% yield over two steps, 98% pure by LC-MS and ¹H-nmr). 2.5 min LC-MS (UV@215 nm: retention time=1.11 min., peak area=100%, TOF-ES⁺ with 25 eV cone voltage: m/z=502 (100%) & 504 (40%)). ¹H NMR: ([D₆]-DMSO, δ in ppm): 10.91 (1H, s), 8.45 (1H, t, J=5.95 Hz), 7.79 (1H, d, J=2.20 Hz), 7.63 (1H, dd, J¹=8.69 Hz, J²=2.20 Hz), 7.17 (2H, d, J=8.69 Hz), 7.05 (1H, d, J=8.78 Hz), 6.86 (2H, d, J=8.69 Hz), 4.98 (1H, br s), 4.70 (2H, s), 4.27 (2H, d, J=5.95 Hz), 3.90 (1H, m), 3.83 (2H, m), 2.91 (2H, t, J=8.28 Hz), 2.70 (1H, m), 2.65 (1H, m), 2.56-2.51 (1H, m), 2.42 (2H, t, J=8.28 Hz), 0.97 (6H, dd, J¹=6.22 Hz, J²=1.74 Hz).

### Synthesis of N-[4-(3-tert-Butylamino-2-hydroxy-propoxy)-benzyl]-2-[2-chloro-4-(6-oxo-1,4,5,6-tetrahydro-pyridazin-3-yl)-phenoxy]-acetamide (17b)

17b was synthesized from 16a using the procedure described for 17a. In the final reaction step tert-butylamine was used instead of isopropylamine. N-[4-(3-tert-butylamino-2-hydroxy-propoxy)-benzyl]-2-[2-chloro-4-(6-oxo-1,4,5,6-tetrahydro-pyridazin-3-yl)-phenoxy]-acetamide (17b) was isolated as colorless powder (184 mg, 26% yield over two steps, 98% pure by LC-MS and ¹H-nmr). 2.5 min LC-MS (UV@215 nm: retention time=1.11 min., peak area=100%, TOF-ES⁺ with 25 eV cone voltage: m/z=516 (100%) & 518 (40%)). ¹H NMR: ([D₆]-DMSO, δ in ppm): 10.90 (1H, s), 8.45 (1H, t, J=5.99 Hz), 7.79 (1H, d, J=2.20 Hz), 7.63 (1H, dd, J¹=8.69 Hz, J²=2.29 Hz), 7.17 (2H, d, J=8.69 Hz), 7.05 (1H, d, J=8.87 Hz), 6.87 (2H, d, J=8.69 Hz), 4.92 (1H, br s), 4.70 (2H, s), 4.26 (2H, d, J=5.95 Hz), 3.93 (1H, m), 3.83 (1H, m), 3.76 (1H, m), 2.91 (2H, t, J=8.23 Hz), 2.67-2.51 (2H, m), 2.42 (2H, t, J=8.28 Hz), 1.01 (9H, s).

### Synthesis of 2-[2-Chloro-4-(6-oxo-1,4,5,6-tetrahydro-pyridazin-3-yl)-phenoxy]-N-{2-[4-(2-hydroxy-3-isopropylamino-propoxy)-phenyl]-ethyl}-acetamide (17c)

17c was synthesized from 16b using the procedure described for 12a. 2-[2-Chloro-4-(6-oxo-1,4,5,6-tetrahydro-pyridazin-3-yl)-phenoxy]-N-{2-[4-(2-hydroxy-3-isopropylamino-propoxy)-phenyl]-ethyl}-acetamide (17c) was isolated as colorless powder (140 mg, 25% yield over two steps, 98% pure by LC-MS and ¹H-nmr). 2.5 min LC-MS (UV@215 nm: retention time=1.02 min., peak area=100%, TOF-ES⁺ with 25 eV cone voltage: m/z=517 (100%) & 519 (40%)). ¹H NMR: (CDCl₃, TMS as internal standard, δ in ppm): 8.88 (1H, br s), 7.80 (1H, d, J=2.20 Hz), 7.54 (1H, dd, J¹=8.60 Hz, J²=2.20 Hz), 7.07 (2H, d, J=8.60 Hz), 6.82 (3H, m), 6.70 (1H, br t), 4.54 (2H, s), 4.07 (1H, m), 3.97 (2H, m), 3.62 (2H, m), 2.97-2.87 (4H, m), 2.82-2.75 (3H, m), 2.61 (2H, t, J=8.23 Hz), 1.14 (6H, d, J=6.31 Hz).

### Synthesis of N-{2-[4-(3-tert-Butylamino-2-hydroxy-propoxy)-phenyl]-ethyl}-2-[2-chloro-4-(6-oxo-1,4,5,6-tetrahydro-pyridazin-3-yl)-phenoxy]-acetamide (17d)

17d was synthesized from 16b using the procedure described for 17a. In the final reaction step tert-butylamine was used instead of iso-propylamine. N-{2-[4-(3-tert-Butylamino-2-hydroxy-propoxy)-phenyl]-ethyl}-2-[2-chloro-4-(6-oxo-1,4,5,6-tetrahydro-pyridazin-3-yl)-phenoxy]-acetamide (17d) was isolated as light yellow foam (122 mg, 21% yield over two steps, 95% pure by LC-MS and ¹H-nmr). 2.5 min LC-MS (UV@215 nm: retention time=1.06 min., peak area=95%, TOF-ES⁺ with 25 eV cone voltage: m/z=531 (100%) & 533 (40%)). ¹H NMR: (CDCl₃, TMS as internal standard, δ in ppm): 8.91 (1H, br s), 7.80 (1H, d, J=2.20 Hz), 7.55 (1H, dd, J¹=8.68 Hz, J²=2.32 Hz), 7.08 (2H, d, J=8.56 Hz), 6.83 (3H, m), 6.72 (1H, br t), 4.54 (2H, s), 4.01-3.93 (3H, m), 3.62 (2H, m), 2.95 (2H, t, J=8.31 Hz), 2.91-2.86 (1H, m), 2.81 (2H, t, J=6.85 Hz), 2.73-2.69 (1H, m), 2.61 (2H, t, J=8.19 Hz), 1.15 (9H, s).

### Example 4

### Synthesis of N-{2-[4-((S)-3-tert-butylamino-2-hydroxy-propoxy)-phenyl]-ethyl}-2-[4-(6-oxo-1,4,5,6-tetrahydro-pyridazin-3-yl)-phenoxy]-acetamide (21)

N-{2-[4-((S)-3-tert-Butylamino-2-hydroxy-propoxy)-phenyl]-ethyl}-2-[4-(6-oxo-1,4,5,6-tetrahydro-pyridazin-3-yl)-phenoxy]-acetamide (21) was synthesized according to Scheme IV.

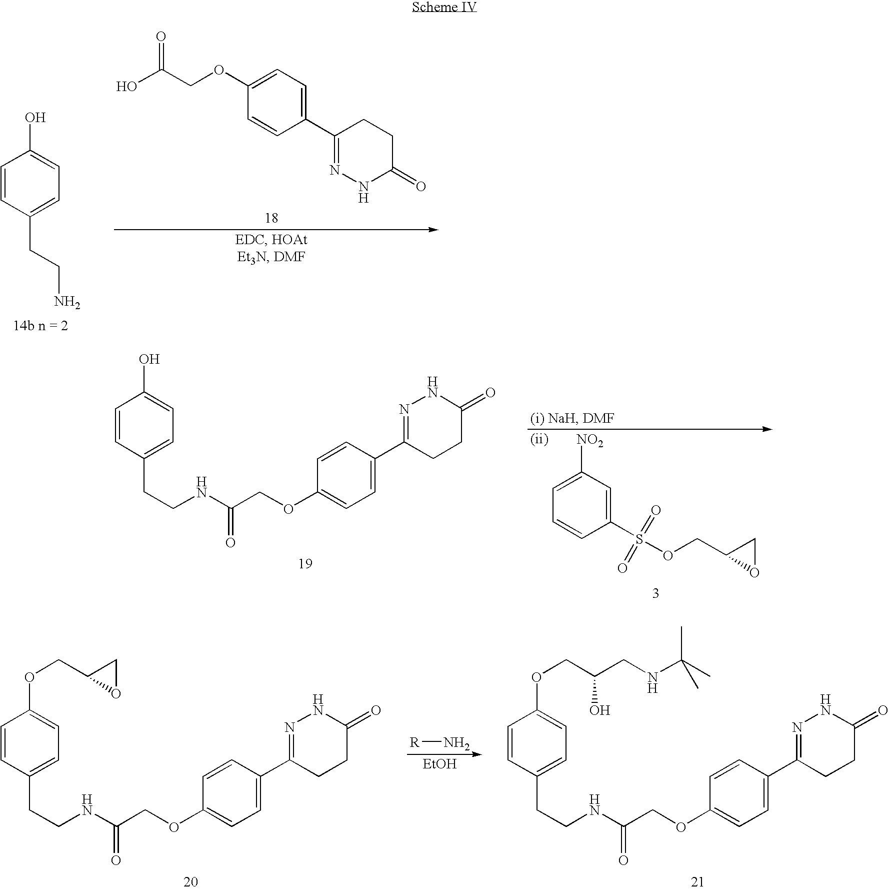

### Synthesis of [4-(6-oxo-1,4,5,6-tetrahydro-pyridazin-3-yl)-phenoxy]-acetic acid (18)

(18) was synthesized using the same procedure as for [2-chloro-4-(6-oxo-1,4,5,6-tetrahydro-pyridazin-3-yl)-phenoxy]-acetic acid (6) except phenol was used in the first step instead of 2-chlorophenol. (18) was obtained as a light yellow powder (13.4 g, 30% overall yield, 99% pure by LCMS and ¹H NMR).

### Synthesis of N-[2-[4-((S)-3-tert-Butylamino-2-hydroxy-propoxy)-phenyl]-ethyl]-2-[4-(6-oxo-1,4,5,6-tetrahydro-pyridazin-3-yl)-phenoxy]-acetamide (21)

21 was synthesized using the same procedure as for N-{2-[4-(3-tert-butylamino-2-hydroxy-propoxy)-phenyl]-ethyl}-2-[2-chloro-4-(6-oxo-1,4,5,6-tetrahydro-pyridazin-3-yl)-phenoxy]-acetamide (17d) except [4-(6-oxo-1,4,5,6-tetrahydro-pyridazin-3-yl)-phenoxy]-acetic acid (13) was used in the first step instead of [2-chloro-4-(6-oxo-1,4,5,6-tetrahydro-pyridazin-3-yl)-phenoxy]-acetic acid (6). 98 mg of 21 was obtained as an off-white powder (overall yield=16%), 100% pure by LCMS and ¹H NMR. 2.5 min LC-MS (UV@215 nm: retention time=1.60 min., peak area=100%, TOF-ES⁺ with 25 eV cone voltage: m/z=497.29 (100%) & 498.30 (30%)). ¹H NMR: (CDCl₃, TMS as internal standard, δ in ppm): 8.88 (1H, br s), 7.67 (2H, m), 7.02 (1H, m), 6.83 (4H, m), 6.48 (1H, m), 4.50 (2H, s), 4.02-3.92 (3H, m), 3.57 (2H, q, J=6.4 Hz), 2.97 (2H, t, J=8.4 Hz), 2.88 (1H, m), 2.78 (2H, t, J=8.4 Hz), 2.69 (1H, m), 2.61 (2H, t, J=8.4 Hz), 1.14 (9H, s).

### Example 5

### Synthesis of the Pyridazinone Glycol (27)

Pyridazinone glycol was synthesized according to Scheme V.

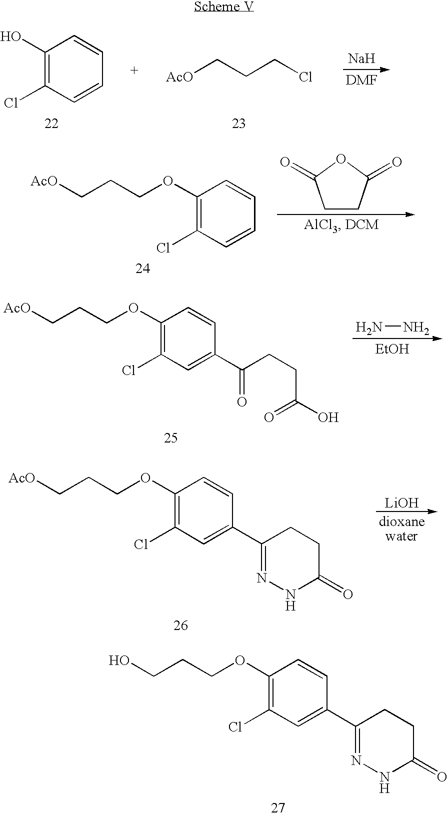

### Synthesis of acetic acid 3-(2-chloro-phenoxy)-propyl ester (24)

To a stirred suspension of sodium hydride (60% dispersion in mineral oil, 7.40 g, 185 mmol) in N,N-dimethylformamide (150 mL) under nitrogen was added portionwise a solution of 2-chlorophenol (22, 16.0 mL, 154 mmol) in N,N-dimethylformamide (50 mL) at 0° C. The reaction mixture was stirred for 30 min at ambient temperature and a solution of acetic acid 3-chloro-propyl ester (23, 21.0 mL, 170 mmol) in N,N-dimethylformamide (50 mL) was added. The reaction mixture was stirred for 30 min at ambient temperature and then for 16 h at 50° C. After cooling to ambient temperature, the reaction mixture was poured into a mixture of ice and saturated aqueous ammonium chloride solution (250 mL) and extracted with ethyl acetate (4×100 mL). The combined organic layers were washed with aqueous sodium hydroxide solution (1 N, 100 mL) and brine (2×100 mL), dried (MgSO₄) and evaporated to dryness to give acetic acid 3-(2-chloro-phenoxy)-propyl ester (24) as light orange oil (31.8 g, 90% yield, 93% pure by LC-MS and ¹H-NMR).

### Synthesis of 4-[4-(3-Acetoxy-propoxy)-3-chloro-phenyl]-4-oxo-butyric acid (25)

To a stirred solution of acetic acid 3-(2-chloro-phenoxy)-propyl ester (24, 31.8 g, 139 mmol) in dichloromethane (100 mL) at ambient temperature under nitrogen was added succinic anhydride (20.8 g, 208 mmol). The reaction mixture was cooled in ice-water and aluminum trichloride (55.6 g, 417 mmol) was added portionwise whilst maintaining the temperature below 20° C. The yellow suspension was stirred at ambient temperature for 20 min and then at 50° C. for 16 h. The obtained dark purple highly viscous oil was allowed to cool to ambient temperature and then carefully hydrolysed with ice-water (100 ml) and ice-aqueous hydrochloric acid (10 N, 100 ml). The aqueous layer was extracted with ethyl acetate (5×100 mL). The combined organic layers were washed with saturated brine (2×100 mL), dried (Na₂SO₄), and concentrated under reduced pressure to give an orange oil. The residue was re-dissolved in hot ethyl acetate (50 mL), hexane (200 mL) was added and the mixture was shaken for 10 min. After standing at ambient temperature for 1 h, the supernatant was decanted. The residue was rinsed with 100 mL hexane and dried under reduced pressure at 50° C. to give 4-[4-(3-acetoxy-propoxy)-3-chloro-phenyl]-4-oxo-butyric acid (25) as a yellow gum (42.7 g, 93% yield, 90% pure by LC-MS and ¹H-NMR).

### Synthesis of Acetic acid 3-[2-chloro-4-(6-oxo-1,4,5,6-tetrahydro-pyridazin-3-yl)-phenoxy]-propyl ester (26)

To a stirred suspension of 4-[4-(3-acetoxy-propoxy)-3-chloro-phenyl]-4-oxo-butyric acid (25, 42.7 g, 130 mmol) in ethanol (300 mL) at 0° C. was added a solution of hydrazine monohydrate (5.74 mL, 117 mmol) in ethanol (50 mL). The reaction mixture was allowed to warm to ambient temperature and stirred at this temperature for 15 min before being heated to reflux and stirred at this temperature for 3 h. Ethyl acetate (60 mL) was added to the hot solution and the mixture was allowed to cool to ambient temperature. The precipitate which formed was filtered off and washed with water (2×100 mL) and cold ethanol (2×100 mL) then dried with suction and then under high vacuum to give acetic acid 3-[2-chloro-4-(6-oxo-1,4,5,6-tetrahydro-pyridazin-3-yl)-phenoxy]-propyl ester (26) as light yellow powder (24.5 g, 58% yield, 97% pure by LC-MS and ¹H-NMR).

### Synthesis of 6-[3-Chloro-4-(3-hydroxy-propoxy)-phenyl]-4,5-dihydro-2H-pyridazin-3-one (27)

To a stirred suspension of acetic acid 3-[2-chloro-4-(6-oxo-1,4,5,6-tetrahydro-pyridazin-3-yl)-phenoxy]-propyl ester (26, 24.5 g, 75.4 mmol) in 1,4-dioxane (125 mL) at ambient temperature were added water (125 mL) and lithium hydroxide (12.7 g, 302 mmol). The reaction mixture was stirred at ambient temperature for 3 h and then acidified to pH 1-2 with aqueous hydrochloric acid (5 N, 100 mL) with stirring. After standing at ambient temperature for 1 h, the precipitate was filtered off and washed with water (2×100 mL) and cold ethanol (2×100 mL). The solid was dried under reduced pressure at 45° C. to give 6-[3-chloro-4-(3-hydroxy-propoxy)-phenyl]-4,5-dihydro-2H-pyridazin-3-one (27) as off-white powder (19.2 g, 90% yield, 99% pure by LC-MS and ¹H-NMR).

### Example 6

### Synthesis of 6-(3-Chloro-4-{3-[4-(2-hydroxy-3-isopropylamino-propoxy)-benzyloxy]-propoxy}-phenyl)-4,5-dihydro-2H-pyridazin-3-one and 6-(4-{3-[4-(3-tert-Butylamino-2-hydroxy-propoxy)-benzyloxy]-propoxy}-3-chloro-phenyl)-4,5-dihydro-2H-pyridazin-3-one (31a and 31b)

6-(3-Chloro-4-{3-[4-(2-hydroxy-3-isopropylamino-propoxy)-benzyloxy]-propoxy}-phenyl)-4,5-dihydro-2H-pyridazin-3-one and 6-(4-{3-[4-(3-tert-Butylamino-2-hydroxy-propoxy)-benzyloxy]-propoxy}-3-chloro-phenyl)-4,5-dihydro-2H-pyridazin-3-one (31a and 31b) were synthesized according to Scheme VI.

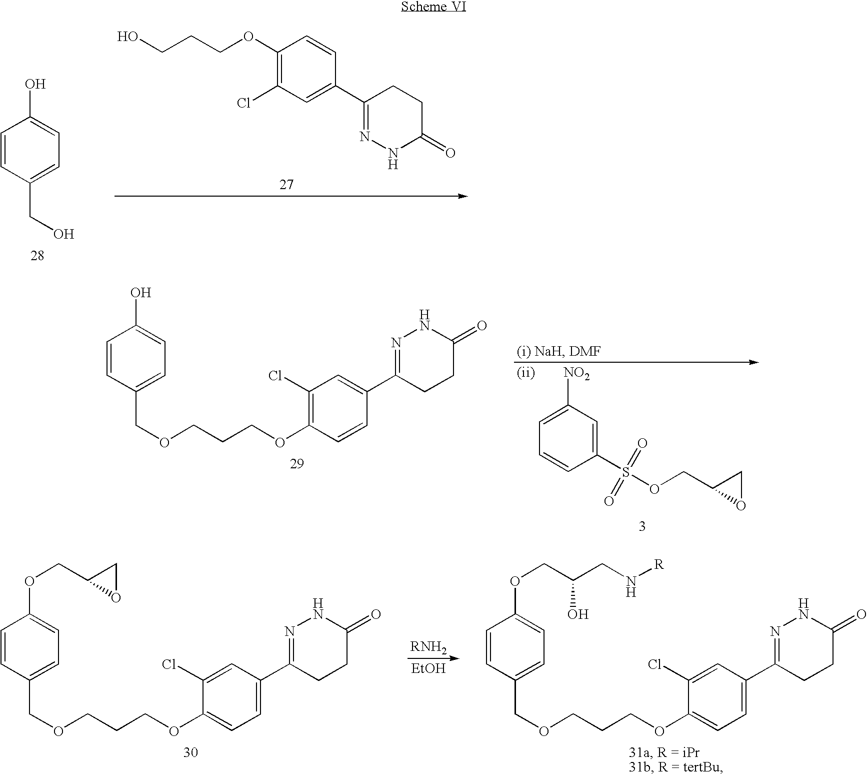

### Synthesis of 6-[3-Chloro-4-[3-(4-hydroxy-benzyloxy)-propoxy]-phenyl]-4,5-dihydro-2H-pyridazin-3-one (29)

To a stirred solution of 4-hydroxymethyl-phenol (28, 220 mg, 1.77 mmol) and 6-[3-chloro-4-(3-hydroxy-propoxy)-phenyl]-4,5-dihydro-2H-pyridazin-3-one (27, 500 mg, 1.77 mmol) in acetonitrile (10 mL) was added ytterbium triflate (11 mg, 0.02 mmol). The mixture was stirred for 4 h under reflux and the solvent was then removed under reduced pressure. H₂O (50 mL) and saturated brine (50 mL) were added and the aqueous phase was extracted with ethyl acetate (3×50 mL). The combined organic layers were washed with saturated brine (50 mL), dried over magnesium sulphate and evaporated to dryness. The residue was dry-loaded onto silica gel (1 g) from ethyl acetate and purified by flash chromatography on silica gel (30 g) eluting with ethyl acetate/heptane 80:20 to give 6-{3-chloro-4-[3-(4-hydroxy-benzyloxy)-propoxy]-phenyl}-4,5-dihydro-2H-pyridazin-3-one (29) as a colorless powder (226 mg, 33% yield, >95% pure by LC-MS and ¹H-nmr).

### Synthesis of 6-(3-Chloro-4-{3-[4-((S)-1-oxiranylmethoxy)-benzyloxy]-propoxy}-phenyl)-4,5-dihydro-2H-pyridazin-3-one (30)

To a stirred suspension of sodium hydride (60% dispersion in mineral oil, 26 mg, 0.650 mmol) in N,N-dimethylformamide (4 mL) under N₂ at 0° C. was added a solution of 6-{3-chloro-4-[3-(4-hydroxy-benzyloxy)-propoxy]-phenyl}-4,5-dihydro-2H-pyridazin-3-one (29, 226 mg, 0.581 mmol) in N,N-dimethylformamide (2 mL) and the reaction mixture was stirred at ambient temperature for 20 min. A solution of (2S)-3-nitro-benzenesulfonic acid oxiranylmethyl ester (3, 151 mg, 0.581 mmol) in N,N-dimethylformamide (2 mL) was then added at 0° C. The reaction mixture was stirred at ambient temperature for 16 h, poured onto a mixture of ice-water (15 mL) and saturated aqueous ammonium chloride solution (15 mL) and extracted with ethyl acetate (3×30 mL). The combined organic layers were washed with aqueous 1N sodium hydroxide solution (2×30 mL), 50% aqueous saturated brine (2×30 mL), and saturated brine (30 ml), dried (Na₂SO₄) and concentrated under reduced pressure to give 6-{3-chloro-4-[3-(4-oxiranylmethoxy-benzyloxy)-propoxy]-phenyl}-4,5-dihydro-2H-pyridazin-3-one (30) as a pale yellow viscous oil, which was used for the next reaction step without further purification.

### Synthesis of 6-(3-Chloro-4-[3-[4-(2-hydroxy-3-isopropylamino-propoxy)-benzyloxy]-propoxy]-phenyl)-4,5-dihydro-2H-pyridazin-3-one (31a)

To a stirred solution of crude 6-{3-chloro-4-[3-(4-oxiranylmethoxy-benzyloxy)-propoxy]-phenyl}-4,5-dihydro-2H-pyridazin-3-one (30) from the last reaction step in ethanol (6 mL) was added iso-propylamine (500 μL, 5.81 mmol). The mixture was stirred for 3 h under reflux and the solvent was then removed under reduced pressure. The residue was purified by flash chromatography on silica gel (8 g) eluting with a gradient of 5-10% methanol in dichloromethane. 6-(3-Chloro-4-{3-[4-(2-hydroxy-3-isopropylamino-propoxy)-benzyloxy]-propoxy}-phenyl)-4,5-dihydro-2H-pyridazin-3-one (31a) was obtained as a colorless foam (144 mg, 49% yield over two steps, 97% pure by LC-MS and ¹H-nmr). 10 min LC-MS (UV@215 nm: retention time=4.53 min., peak area=100%, TOF-ES⁺ with 25 eV cone voltage: m/z=504 (100%) & 506 (40%)). ¹H NMR: ([D₆]-DMSO, δ in ppm): 10.89 (1H, s), 7.76 (1H, d, J=2.20 Hz), 7.65 (1H, dd, J¹=8.69 Hz, J²=2.20 Hz), 7.18 (3H, m), 6.86 (2H, d, J=8.69 Hz), 4.38 (2H, s), 4.15 (2H, t, J=6.22 Hz), 3.90 (1H, m), 3.83 (2H, m), 3.56 (2H, t, J=6.17 Hz), 2.90 (2H, t, J=8.23 Hz), 2.76-2.65 (2H, m), 2.58-2.52 (1H, m), 2.41 (2H, t, J=8.23 Hz), 1.99 (2H, m), 1.98 (6H, dd, J¹=6.22 Hz, J²=1.37 Hz).

### Synthesis of 6-(4-{3-[4-(3-tert-Butylamino-2-hydroxy-propoxy)-benzyloxy]-propoxy}-3-chloro-phenyl)-4,5-dihydro-2H-pyridazin-3-one (31b)

31b was synthesized via the procedure described for 31a using tert-butylamine instead of iso-propylamine in the last reaction step. A pale yellow gum (110 mg, 29% yield over last 2 steps), 98% pure by LC-MS and ¹H-nmr) was obtained. 2.5 min LC-MS (UV@215 nm: retention time=1.49 min., peak area=100%, TOF-ES⁺ with 25 eV cone voltage: m/z=518 (100%) & 520 (40%)). ¹H NMR: ([D₆]-DMSO, δ in ppm): 10.89 (1H, s), 7.77 (1H, d, J=2.20 Hz), 7.64 (1H, dd, J¹=8.56 Hz, J²=2.20 Hz), 7.19 (3H, m), 6.86 (2H, d, J=8.56 Hz), 4.38 (2H, s), 4.15 (2H, t, J=6.24 Hz), 3.94 (1H, m), 3.82 (1H, m), 3.76 (1H, m), 3.56 (2H, t, J=6.24 Hz), 2.90 (2H, t, J=8.19 Hz), 2.65-2.51 (2H, m), 2.41 (2H, t, J=8.31 Hz), 1.99 (2H, m), 1.01 (9H, s).

### Example 7

### Synthesis of C-2 Linked Analogues

C-2 linked analogues were synthesized according to Scheme VII.

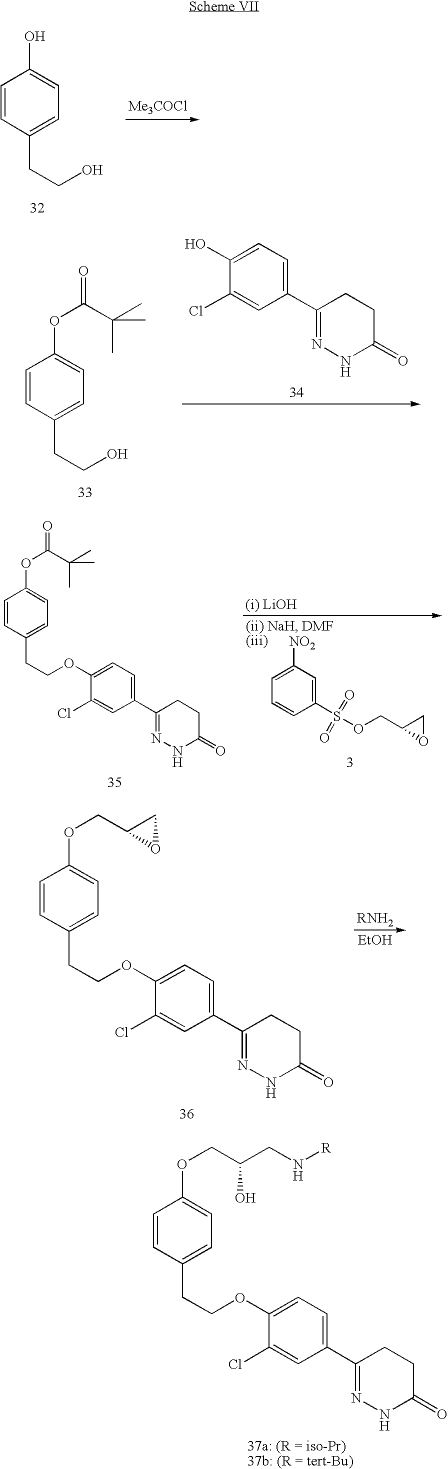

### Synthesis of 2,2-Dimethyl-propionic acid 4-(2-hydroxy-ethyl)-phenyl ester (33)

To a stirred solution of 4-(2-hydroxy-ethyl)-phenol (32, 5.0 g, 36.2 mmol) in dichloromethane (50 mL) was added triethylamine (10.1 mL, 72.4 mmol). A solution of pivaloyl chloride (4.41 g, 36.2 mmol) in dichloromethane (10 mL) was added dropwise at 0° C. and the mixture was then stirred for 16 h at ambient temperature. The mixture was poured into ice-water (100 mL), the phases were separated and the aqueous phase was extracted with dichloromethane (100 mL). The combined organic layers were dried over magnesium sulphate and evaporated to dryness. The residue was purified by flash chromatography on silica gel (200 g) eluting with 10-25% ethyl acetate in heptane. 2,2-Dimethyl-propionic acid 4-(2-hydroxy-ethyl)-phenyl ester (33) was obtained as a colorless solid (6.5 g, 81% yield, 95% pure by LC-MS and ¹H-NMR).

### Synthesis of 2,2-Dimethyl-propionic acid 4-{2-[2-chloro-4-(6-oxo-1,4,5,6-tetrahydro-pyridazin-3-yl)-phenoxy]-ethyl}-phenyl ester (35)

A suspension of 2,2-dimethyl-propionic acid 4-(2-hydroxy-ethyl)-phenyl ester (33, 297 mg, 1.34 mmol), 6-(3-chloro-4-hydroxy-phenyl)-4,5-dihydro-2H-pyridazin-3-one (34, 250 mg, 1.10 mmol) and polymer-supported triphenylphosphine (1.48 g, 2.23 mmol) in dichloromethane (40 mL) was vigorously stirred for 10 min at ambient temperature. To the suspension was added diisopropyl azodicarboxylate (285 μL, 1.45 mmol) in one portion. The mixture was stirred for 18 h at ambient temperature and then filtered. The filter residue was rinsed with dichloromethane (3×20 mL). The combined filtrates were evaporated to dryness. The residue was purified by flash chromatography on silica gel (100 g) eluting with a gradient of 20-60% ethyl acetate in heptane to give 2,2-dimethyl-propionic acid 4-{2-[2-chloro-4-(6-oxo-1,4,5,6-tetrahydropyridazin-3-yl)-phenoxy]-ethyl}-phenyl ester (35) as a colorless powder (840 mg, 74% yield, >95% pure by LC-MS and ¹H-nmr).

### Synthesis of 6-{3-Chloro-4-[2-(4-oxiranylmethoxy-phenyl)-ethoxy]-phenyl}-4,5-dihydro-2H-pyridazin-3-one (36)

To a stirred solution of 4-{2-[2-chloro-4-(6-oxo-1,4,5,6-tetrahydropyridazin-3-yl)-phenoxy]-ethyl}-phenyl ester (35, 840 mg, 1.96 mmol) in tetrahydrofurane (25 mL) and water (25 mL) was added lithium hydroxide (329 mg, 7.83 mmol). The mixture was stirred for 16 h at ambient temperature, acidified to pH 6 with 1N aqueous hydrochloric acid and extracted with tert-butylmethylether (3×50 mL). The combined organic layers were dried over sodium sulphate and evaporated to dryness. The residue was dissolved in butan-2-one (20 mL) and (S)-3-nitro-benzenesulfonic acid oxiranylmethyl ester (3, 480 mg, 1.85 mmol) and potassium carbonate (250 mg, 1.85 mmol) were added. The mixture was stirred for 16 h under reflux and the solvent was then removed under reduced pressure. The residue was re-dissolved in dichloromethane (60 mL), washed with aqueous sodium hydroxide solution (1N, 2×50 mL) and saturated brine (50 mL), dried over sodium sulphate and evaporated to dryness. 6-{3-Chloro-4-[2-(4-oxiranylmethoxy-phenyl)-ethoxy]-phenyl}-4,5-dihydro-2H-pyridazin-3-one (36) was obtained as a yellow viscous oil, which was used in the next reaction step without further purification.

### Synthesis of 6-(3-Chloro-4-{2-[4-(2-hydroxy-3-isopropylamino-propoxy)-phenyl]-ethoxy}-phenyl)-4,5-dihydro-2H-pyridazin-3-one (37a)

Crude 6-{3-chloro-4-[2-(4-oxiranylmethoxy-phenyl)-ethoxy]-phenyl}-4,5-dihydro-2H-pyridazin-3-one (36) from the last step was dissolved in ethanol (15 mL) and iso-propylamine (1.5 mL, 17.5 mmol) was added. The mixture was heated under reflux for 4 h and the solvent was then removed under reduced pressure. The residue was purified by flash chromatography on silica gel (8 g) eluting with dichloromethane/methanol 10:1 to give 6-(3-chloro-4-{2-[4-(2-hydroxy-3-isopropylamino-propoxy)-phenyl]-ethoxy}-phenyl)-4,5-dihydro-2H-pyridazin-3-one (37a) as a pale yellow foam (279 mg, 31% yield over three steps, 99% pure by LC-MS and ¹H-nmr). 2.5 min LC-MS (UV@215 nm: retention time=1.25 min., peak area=100%, TOF-ES⁺ with 25 eV cone voltage: m/z=460 (100%) & 462 (50%)). ¹H NMR: (CDCl₃, TMS as internal standard, δ in ppm): 8.64 (1H, s), 7.77 (1H, d, J=2.20 Hz), 7.53 (1H, dd, J¹=8.56 Hz, J²=2.20 Hz), 7.24 (2H, d, J=8.66 Hz), 6.88 (3H, m), 4.20 (2H, t, J=6.85 Hz), 4.05-3.99 (1H, m), 3.96 (2H, m), 3.11 (2H, t, J=6.85 Hz), 2.93 (2H, m), 2.89-2.81 (1H, m), 2.76-2.71 (1H, m), 2.59 (2H, m), 2.23 (2H, br s), 1.10 (6H, d, J=6.36 Hz).

### Synthesis of 6-(4-{2-[4-(3-tert-Butylamino-2-hydroxy-propoxy)-phenyl]-ethoxy}-3-chloro-phenyl)-4,5-dihydro-2H-pyridazin-3-one (37b)

37b was synthesized via the procedure described for 37a using tert-butylamine instead of iso-propylamine in the last reaction step. A pale yellow foam (200 mg, 47% yield over last 2 steps), 99% pure by LC-MS and ¹H-nmr was obtained. 2.5 min LC-MS (UV@215 nm: retention time=1.15 min., peak area=100%, TOF-ES⁺ with 25 eV cone voltage: m/z=474 (100%) & 476 (40%)). ¹H NMR: (CDCl₃, TMS as internal standard, δ in ppm): 8.63 (1H, s), 7.77 (1H, d, J=2.45 Hz), 7.53 (1H, dd, J¹=8.56 Hz, J²=2.20 Hz), 7.24 (2H, d, J=8.56 Hz), 6.88 (3H, m), 4.21 (2H, t, J=6.97 Hz), 4.02-3.92 (3H, m), 3.11 (2H, t, J=6.85 Hz), 2.93 (2H, t, J=8.19 Hz), 2.89-2.83 (1H, m), 2.71-2.65 (1H, m), 2.59 (2H, t, J=8.19 Hz), 2.13 (2H, br s), 1.12 (9H, s).

### Example 8

### Synthesis of 6-[3-Chloro-4-(3-{2-[4-((S)-2-hydroxy-3-isopropylamino-propoxy)-phenyl]-ethoxy}-propoxy)-phenyl]-4,5-dihydro-2H-pyridazin-3-one and 6-[4-(3-{2-[4-((S)-3-tert-Butylamino-2-hydroxy-propoxy)-phenyl]-ethoxy}-propoxy)-3-chloro-phenyl]-4,5-dihydro-2H-pyridazin-3-one (37a and 37b)

6-[3-Chloro-4-(3-{2-[4-((S)-2-hydroxy-3-isopropylamino-propoxy)-phenyl]-ethoxy}-propoxy)-phenyl]-4,5-dihydro-2H-pyridazin-3-one and 6-[4-(3-{2-[4-((S)-3-tert-Butylamino-2-hydroxy-propoxy)-phenyl]-ethoxy}-propoxy)-3-chloro-phenyl]-4,5-dihydro-2H-pyridazin-3-one (46a and 46b) were synthesized according to Scheme VIII.

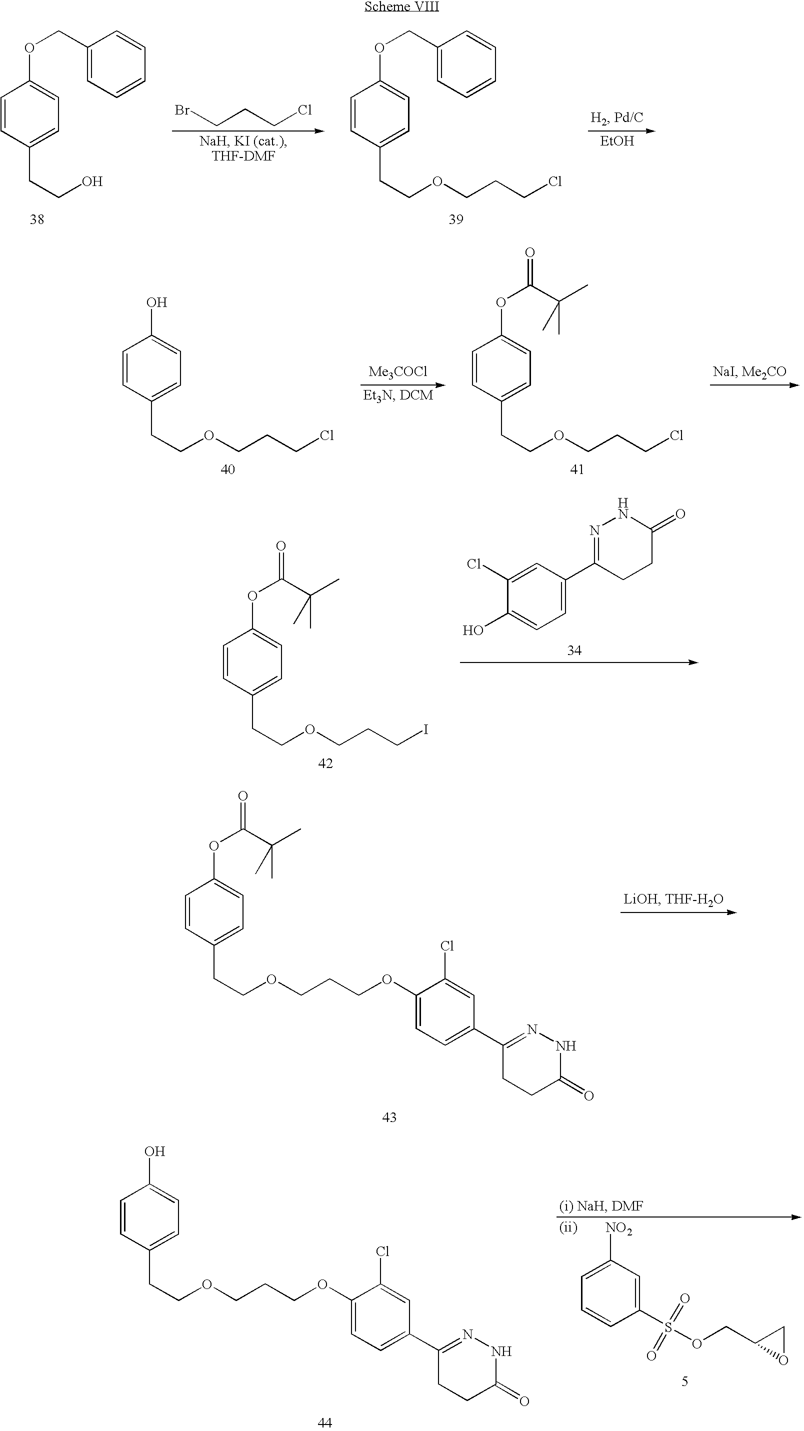

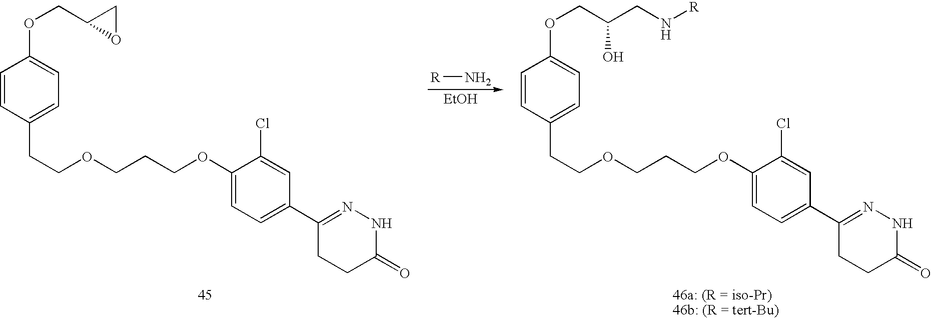

### Synthesis of 1-Benzyloxy-4-[2-(3-chloro-propoxy)-ethyl]-benzene (39)

To a stirred suspension of sodium hydride (60% dispersion in mineral oil) (703 mg, 17.5 mmol) in tetrahydrofuran/N,N-dimethylformamide (1:1) (6 ml) was added slowly a solution of 2-(4-benzyloxyphenyl)ethanol (2.0 g, 8.8 mmol) in tetrahydrofuran (9 ml) under nitrogen, and the resulting reaction mixture was stirred at ambient temperature for 30 minutes. Following this, further portions of tetrahydrofuran (5 ml) and N,N-dimethylformamide (3 ml) were added and the reaction mixture was stirred under nitrogen for a further 3 hours. To the reaction mixture were then added 1-bromo-3-chloropropane (1.7 ml, 17.5 mmol) and potassium iodide (145 mg, 0.88 mmol). The reaction mixture was then heated to 70° C. and stirred at this temperature for 20 h then allowed to cool to ambient temperature. To the reaction mixture were then added tert-butylmethyl ether (50 ml), saturated aqueous ammonium chloride (10 ml) and water (30 ml). The resulting 2-phase mixture was shaken and separated and the aqueous phase was extracted with tert-butylmethyl ether (50 ml). The combined organic extracts were then washed with saturated brine (80 ml), dried (MgSO₄) and concentrated under reduced pressure to give an orange solid. To this solid were added heptanes (3 ml) and ethyl acetate (5 ml) and the mixture was sonicated for 10 minutes then left to stand for 18 h. The solid was then filtered and the filtrate was taken and concentrated under reduced pressure to give a yellow oily solid as the crude product. This product was purified by flash column chromatography over silica gel using gradient eluent 5-10% ethyl acetate in heptanes to afford (39) as a colorless oil (518 mg, 19% yield).

### Synthesis of 4-[2-(3-Chloro-propoxy)-ethyl]-phenol (40)

To a stirred solution of 1-benzyloxy-4-[2-(3-chloro-propoxy)-ethyl]-benzene (39) (518 mg, 1.7 mmol) in ethanol (40 ml) was added 10% Pd on C (181 mg, 0.17 mmol). The resulting stirred suspension was then placed under vacuum then a nitrogen atmosphere (repeated twice) then placed under vacuum then a hydrogen atmosphere (repeated twice). The reaction mixture was then stirred at ambient temperature under a hydrogen atmosphere for 9 h then filtered through a bed of celite and the filtrate was taken and concentrated under reduced pressure to afford (40) as a pale brown oil (287 mg, 79% yield) which was >90% pure by ¹H NMR.

### Synthesis of 2,2-Dimethyl-propionic acid 4-[2-(3-chloro-propoxy)-ethyl]-phenyl ester (41)

To a solution of 4-[2-(3-chloro-propoxy)-ethyl]-phenol (40) (287 mg, 1.33 mmol) in dichloromethane (5 ml) at 0° C. were added triethylamine (0.373 ml, 2.66 mmol) and trimethylacetyl chloride (0.165 ml, 1.33 ml). The reaction mixture was then stirred at ambient temperature for 20 h then dichloromethane (10 ml) and water (10 ml) were added. The resulting 2-phase system was then separated using a hydrophobic filter membrane and the organic solution was taken and concentrated under reduced pressure to afford (41) as a colorless oil (427 mg, 78% pure by LCMS, 83% corrected yield).

### Synthesis of 2,2-Dimethyl-propionic acid 4-[2-(3-Iodo-propoxy)-ethyl]-phenyl ester (42)

To a stirred solution of 2,2-dimethyl-propionic acid 4-[2-(3-chloro-propoxy)-ethyl]-phenyl ester (41) (427 mg, 1.43 mmol) in acetone (15 ml) at ambient temperature was added sodium iodide (1.07 g, 7.14 mmol) then the reaction mixture was heated to reflux and stirred at this temperature for 18 h. To the reaction mixture was then added a further portion of acetone (15 ml) and the reaction mixture was heated to reflux with stirring for a further 24 h. Following this, further portions of sodium iodide (1.07 g, 7.14 mmol) and acetone (25 ml) were added and the reaction mixture was heated to reflux with stirring for a further 24 h. After cooling, the reaction mixture was filtered and the solid which was collected was washed with dichloromethane (10 ml). The filtrate was then taken and concentrated under reduced pressure to afford yellow solid as the crude product, which was purified by flash column chromatography over silica gel using dichloromethane as the eluent. (42) was obtained as a pale yellow oil (430 mg, 78% pure, 60% corrected yield).

### Synthesis of 2,2-Dimethyl-propionic acid 4-[2-{3-[2-chloro-4-(6-oxo-1,4,5,6-tetrahydro-pyridazin-3-yl)-phenoxy]-propoxy}-ethyl)-phenyl ester (43)

6-(3-Chloro-4-hydroxy-phenyl)-4,5-dihydro-2H-pyridazin-3-one (34, 193 mg, 0.86 mmol) and potassium carbonate (130 mg, 0.94 mmol) were dissolved in N,N-dimethylformamide (4 ml) with stirring at ambient temperature and to this solution was added a solution of 2,2-dimethyl-propionic acid 4-[2-(3-iodo-propoxy)-ethyl]-phenyl ester (42) (430 mg, 78% pure, 0.86 mmol) in N₇N-dimethylformamide (4 ml) and the reaction mixture was stirred at ambient temperature for 18 h. To the reaction mixture was then added ethyl acetate (5 ml) and water (30 ml) and the resulting 2-phase system was shaken and separated. The aqueous phase was then extracted with ethyl acetate (3×20 ml) then the combined organic extracts were washed with 2N NaOH aqueous solution (2×25 ml), saturated aqueous brine (3×30 ml), dried (Na₂SO₄) and concentrated under reduced pressure to afford the crude product as a yellow oil. This product was then purified by flash column chromatography over silica gel using 0-10% ethyl acetate in dichloromethane as the eluent. (43) was obtained as a colorless oil (228 mg, 33% yield, >90% pure by LCMS).

### Synthesis of 6-(3-Chloro-4-{3-[2-(4-hydroxy-phenyl)-ethoxy]-propoxy}-phenyl)-4,5-dihydro-2H-pyridazin-3-one (44)

To a stirred solution of 2,2-dimethyl-propionic acid 4-[2-{3-[2-chloro-4-(6-oxo-1,4,5,6-tetrahydro-pyridazin-3-yl)-phenoxy]-propoxy}-ethyl)-phenyl ester (43) (228 mg, 0.47 mmol) in tetrahydrofuran (5 ml) was added a solution of lithium hydroxide (79 mg, 1.88 mmol) in water (5 ml). The reaction mixture was then stirred at ambient temperature for 72 h before tert-butylmethyl ether (10 ml) and the pH was adjusted to pH 6-7 using 1N HCl. The 2-phase system was then shaken and separated and the aqueous phase was extracted with tert-butylmethyl ether (3×20 ml). The combined organic extracts were then dried (MgSO₄) and concentrated under reduced pressure to afford (44) as a yellow oil (205 mg, 97% yield, >90% pure by LCMS).

### 6-(3-Chloro-4-(3-{2-[4-((S)-1-oxiranylmethoxy)-phenyl]-ethoxy}-propoxy)-phenyl]-4,5-dihydro-2H-pyridazin-3-one (45)

(45) was synthesized using the same procedure as for (4) except 6-(3-chloro-4-{3-[2-(4-hydroxy-phenyl)-ethoxy]-propoxy}-phenyl)-4,5-dihydro-2H-pyridazin-3-one (44) was used as the starting material instead of {2-[2-(4-hydroxy-phenyl)-acetylamino]-ethyl}-carbamic acid tert butyl ester (2). (45) was obtained as a yellow oil (224 mg) of purity 61% by LCMS (corrected yield=63%) and was used for the next step without further purification.

### Synthesis of 6-[3-Chloro-4-(3-[2-[4-((S)-2-hydroxy-3-isopropylamino-propoxy)-phenyl]-ethoxy]-propoxy)-phenyl]-4,5-dihydro-2H-pyridazin-3-one (46a)

(46a) was synthesized using the same procedure as for (5) except 6-(3-chloro-4-(3-{2-[4-((S)-1-oxiranylmethoxy)-phenyl]-ethoxy}-propoxy)-phenyl]-4,5-dihydro-2H-pyridazin-3-one (45) was used as the starting material instead of (2-{2-[4-((S)-1-oxiranylmethoxy)-phenyl]-acetylamino}-ethyl)-carbamic acid tert butyl ester (4). The crude product was purified by flash chromatography on silica gel (4 g) eluting with gradient eluent 5-7% methanol in dichloromethane to give 6-[3-Chloro-4-(3-{2-[4-((S)-2-hydroxy-3-isopropylamino-propoxy)-phenyl]-ethoxy}-propoxy)-phenyl]-4,5-dihydro-2H-pyridazin-3-one (46a) as a pale yellow foam (24 mg, 19% yield), 100% pure by LC-MS and ¹H-nmr). 2.5 min LC-MS (UV@215 nm: retention time=1.27 min., peak area=100%, TOF-ES⁺ with 25 eV cone voltage: m/z=518.29 (100%) & 520.30 (50%)). ¹H NMR: (CDCl₃, TMS as internal standard, δ in ppm): 9.06 (1H, s), 7.71 (1H, d, J=2.8 Hz), 7.57 (1H, dd, J¹=9.2 Hz, J²=2.8 Hz), 7.09 (2H, d, J=8.4 Hz), 6.80 (1H, d, J=8.8 Hz), 6.76 (2H, m), 4.05 (3H, m), 3.92 (2H, d, J=5.6 Hz), 3.67-3.61 (4H, m), 2.94 (2H, t, J=8.4 Hz), 2.91-2.70 (4H, m), 2.60 (2H, t, J=8.8 Hz), 2.07 (2H, m), 1.11 (6H, d, J=6.8 Hz).

### Synthesis of 6-[4-(3-{2-[4-((S)-3-tert-Butylamino-2-hydroxy-propoxy)-phenyl]-ethoxy}-propoxy)-3-chloro-phenyl]-4,5-dihydro-2H-pyridazin-3-one (46b)

46b was synthesized via the procedure described for 46a using tert-butylamine instead of iso-propylamine in the last reaction step. 6-[4-(3-{2-[4-((S)-3-tert-butylamino-2-hydroxy-propoxy)-phenyl]-ethoxy}-propoxy)-3-chloro-phenyl]-4,5-dihydro-2H-pyridazin-3-one (46b) was obtained as a pale yellow foam (25 mg, 20% yield), 100% pure by LC-MS and ¹H-nmr). 2.5 min LC-MS (UV@215 nm: retention time=1.86 min., peak area=100%, TOF-ES⁺ with 25 eV cone voltage: m/z=532.31 (100%) & 534.33 (40%)). ¹H NMR: (CDCl₃, TMS as internal standard, δ in ppm): 9.00 (1H, s), 7.71 (1H, d, J=2.8 Hz), 7.57 (1H, dd, J¹=9.2 Hz, J²=2.8 Hz), 7.09 (2H, d, J=8.4 Hz), 6.81 (1H, d, J=8.8 Hz), 6.76 (2H, m), 4.06 (2H, t, J=6.8 Hz), 3.93 (2H, m), 3.66-3.61 (4H, m), 2.94 (2H, t, J=8.4 Hz), 2.84-2.78 (4H, m), 2.69 (1H, dd, J¹=12.0 Hz, J²=8.0 Hz), 2.60 (2H, m), 2.07 (2H, m), 1.14 (9H, s).

### Example 9

### Synthesis of 2′{3-[4-(2-Hydroxy-3-isopropylamino-propoxy)-phenoxy]-propoxy}-2-methyl-6-oxo-1,6-dihydro-[3,4′]bipyridinyl-5-carbonitrile

2′{3-[4-(2-Hydroxy-3-isopropylamino-propoxy)-phenoxy]-propoxy}-2-methyl-6-oxo-1,6-dihydro-[3,4′]bipyridinyl-5-carbonitrile is synthesized according to Scheme IX.

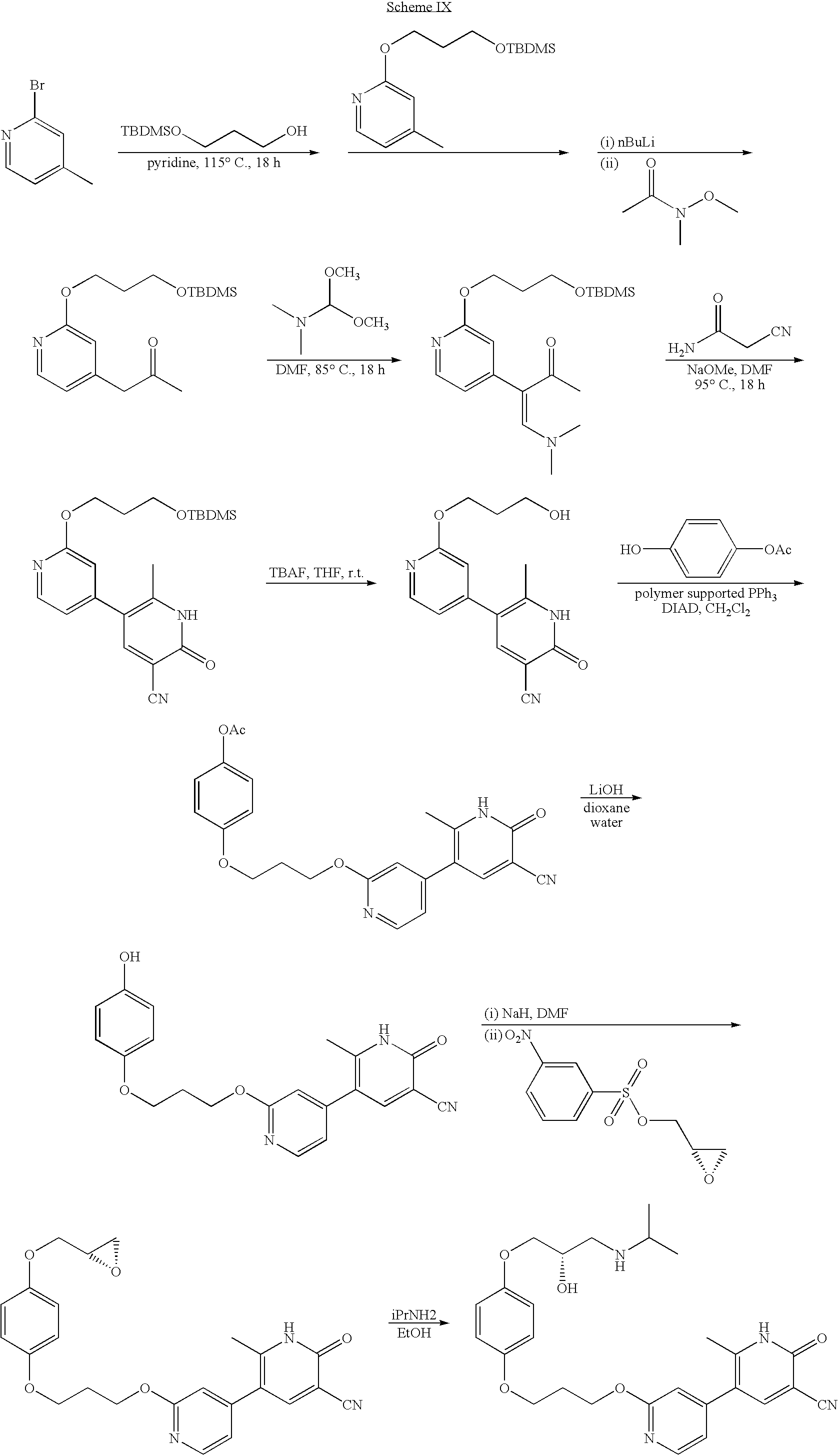

### Example 10

### Synthesis of 6′-{3-[4-2-hydroxy-3-isopropylamino-propoxy)-phenoxy]-propoxy}-2-methyl-6-oxo-1,6-dihydro-[3,3′]bipyridinyl-5-carbonitrile

6′-{3-[4-2-Hydroxy-3-isopropylamino-propoxy)-phenoxy]-propoxy}-2-methyl-6-oxo-1,6-dihydro-[3,3′]bipyridinyl-5-carbonitrile is synthesized according to the method of Scheme X.

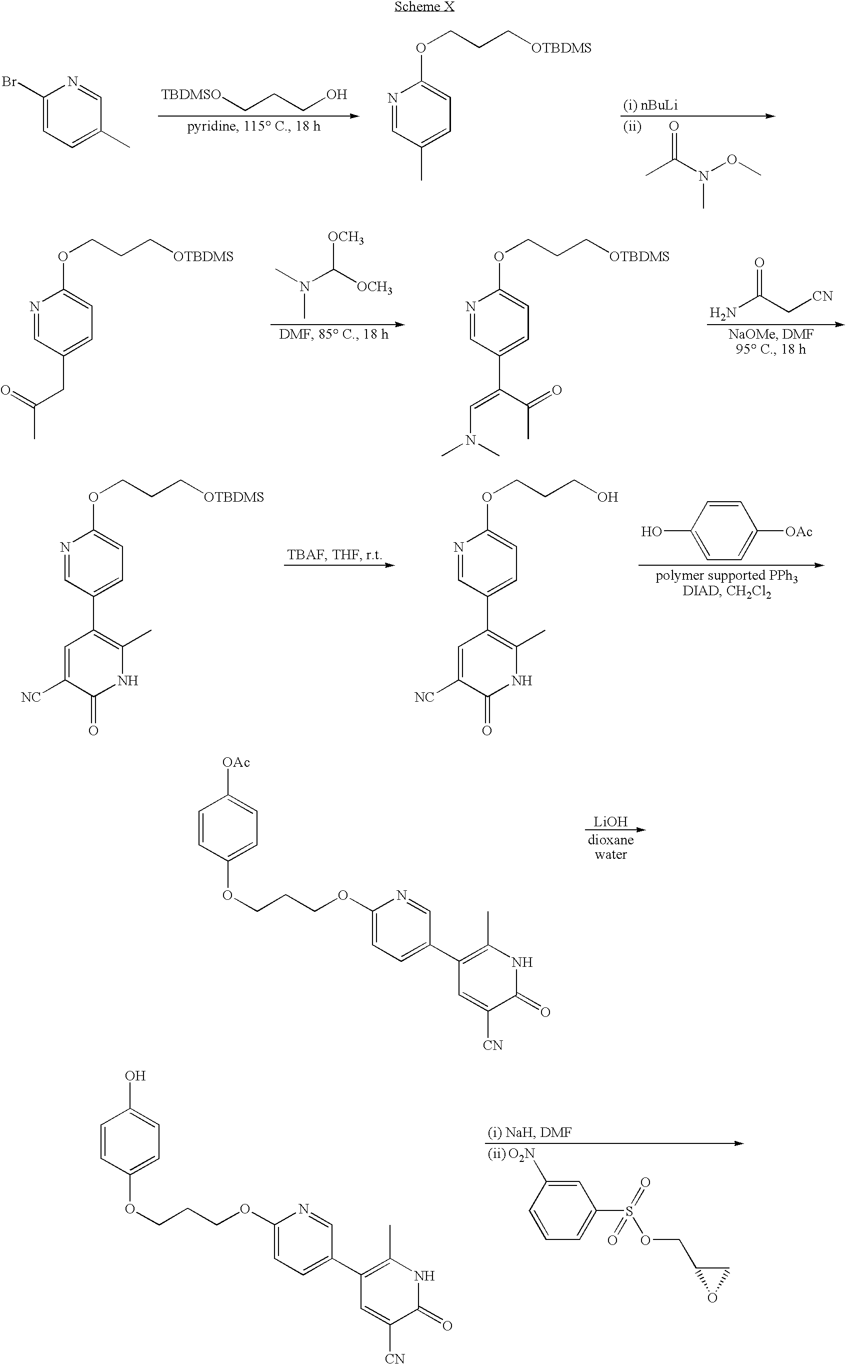

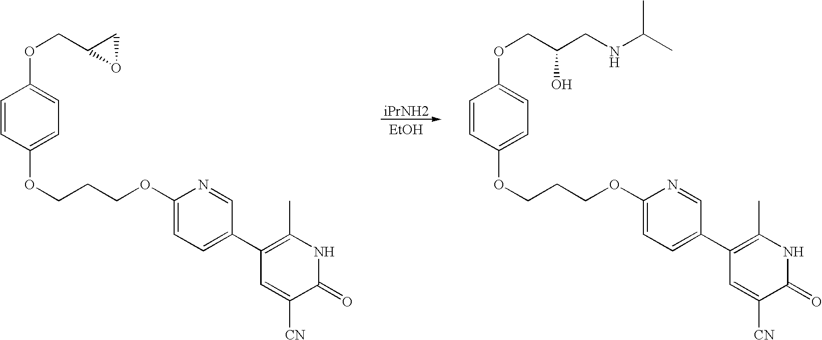

### Example 11

### Synthesis of 6-[3-Chloro-4-(2-{[4-(2-hydroxy-3-isopropylamino-propoxy)-9H-carbazol-1-yl]-methyl-amino]-ethoxy)-phenyl]-4,5-dihydro-2H-pyridazin-3-one; 6-[4-(2-{[4-(3-tert-butylamino-2-hydroxy-propoxy)-9H-carbazol-1-yl]-methyl-amino}-ethoxy)-3-chloro-phenyl]-4,5-dihydro-2H-pyridazin-3-one; and 6-(3-chloro-4-{2-[(4-{2-hydroxyl-3-[2-(2-methoxy-phenoxy)-ethylamino]-propoxy}-9H-carbazol-1-yl)-methyl-amino]-ethoxy}-phenyl)-4,5-dihydro-2H-pyridazin-3-one

6-[3-Chloro-4-(2-{[4-(2-hydroxy-3-isopropylamino-propoxy)-9H-carbazol-1-yl]-methyl-amino]-ethoxy)-phenyl]-4,5-dihydro-2H-pyridazin-3-one; 6-[4-(2-{[4-(3-tert-butylamino-2-hydroxy-propoxy)-9H-carbazol-1-yl]-methyl-amino}-ethoxy)-3-chloro-phenyl]-4,5-dihydro-2H-pyridazin-3-one; and 6-(3-chloro-4-{2-[(4-{2-hydroxyl-3-[2-(2-methoxy-phenoxy)-ethylamino]-propoxy}-9H-carbazol-1-yl)-methyl-amino]-ethoxy}-phenyl)-4,5-dihydro-2H-pyridazin-3-one are synthesized according to the method of Scheme XI.

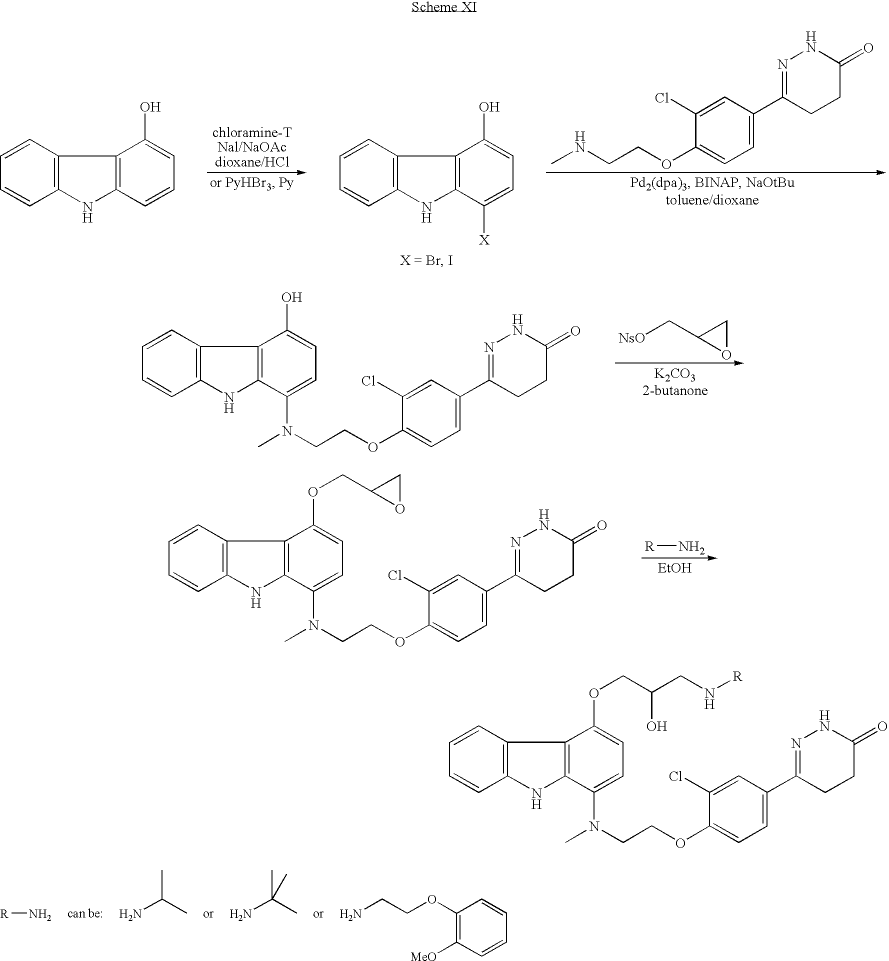

### Example 12

### PDE-3 Inhibitory Activity

In vitro assay for measuring cAMP PDE-3 inhibitory activity Human platelet cyclic AMP phosphodiesterase was prepared according to the method of Alvarez et al., Mol. Pharmacol. 29: 554 (1986). The PDE incubation medium contained 10 mM Tris-HCl buffer, pH 7.7, 10 mM MgSO₄, and 1 μM [³H]AMP (0.2 μCi) in a total volume of 1.0 mL. Test compounds were dissolved in DMSO immediately prior to addition to the incubation medium, and the resulting mixture was allowed to stand for 10 minutes prior to the addition of enzyme. Following the addition of PDE, the contents were mixed and incubated for 10 minutes at 30° C. Three assays each were performed for each of five test compound concentrations, the mean of the determinations (n=3) at each concentration was plotted, and IC₅₀ values were determined graphically. All of the compounds tested, including compounds 7, 12a, 12b, 13, 17a, 17b, 17c, 17d, 21, 31a, 31b, 37a, 37b, 46a, and 46b had PDE3 inhibitory IC₅₀ values less than 1 μM. In addition, compounds 7, 12b, 17c, 17d, 31b, 37a, 37b, 46a, and 46b had PDE3 inhibitory IC₅₀ values less than 100 nM.

### Example 13

### β-Adrenergic Receptor Binding and Blocking Activity

β-Adrenergic receptor binding and blocking activity was evaluated by one or more of the methods below.

Radioligand for Measuring Non-Specific β-Receptor Activity

Non-specific receptor binding was measured for each of the test compounds for beta-receptors from rat cortical membranes, using [³H]DHA as the radioligand, as described in Riva and Creese, Mol. Pharmacol. 36:211 (1989) and Arango et al., Brain Res., 516:113 (1990). The non-specific beta-adrenergic receptor IC₅₀ values for compounds 13, 17a, 17b, 17c, 17d, 21, 31a, 31b, 37a, 37b, 46a, and 46b were less than 1 μM. In addition, the non-specific beta-adrenergic receptor IC₅₀ values for compounds 13, 17d, 31a, 31b, 46a, and 46b were less than 100 nM.

Radioligand for Measuring β₁-Receptor Affinity

β₁-Adrenergic receptor binding was measured in human recombinant beta-1 receptors expressed in CHO-REX16 cells, using [¹²⁵I] (−) Iodocyanopindolol (2000 Ci/mmol) as the radioligand, as described in Kalaria et al., J. Neurochem. 53: 1772-81 (1998), and Minneman et al., Mol. Pharmacol. 16: 34-46 (1979). Compounds 17d, 21, 31b, 46a, and 46b each inhibited greater than 25% β1-adrenergic binding at a concentration of 100 nM.

Radioligand for Measuring β₂-Receptor Affinity

β₂-Adrenergic receptor binding was measured in human recombinant beta-2 receptors expressed in CHO-WT21 cells, using [¹²⁵I] (−) Iodocyanopindolol (2000 Ci/mmol) as the radioligand, as described in Kalaria et al. (1998) and Minneman et al. (1979), supra. Compounds 17d, 21, 31b, 46a, and 46b each inhibited greater than 25% β1-adrenergic binding at a concentration of 100 nM.

Effect on β₂-Adrenergic Blocking Activity

Tracheal chains are prepared as described by Castillo and DeBeer, J. Pharm. Exp. Ther. 90: 104 (1947), suspended in tissue baths maintained at 37° C. containing Tyrodes solution gassed with 95% O₂-5% CO₂, and attached to an isometric force-displacement transducer. After an equilibration period of 2 hours, the preparations are induced to contract with carbachol (3×10⁻⁷ M), and relaxation is induced with cumulative dose response curves for isoproterenol first in the absence of and then in the presence of the test compound. A contact time of 10 minutes is allowed for all test compounds. Affinity constants are determined by comparing the shift in the dose-response curve for each test compound with that of isoproterenol (EC₅₀=2.3×0.2×10⁻⁸ M).

### Example 14

### Restoration of Calcium Homeostasis in Heart Tissue

Effect on Contraction-Relaxation in Guinea Pig Papillary Muscle

Male guinea pigs (400-500 g) are killed by cervical dislocation and the hearts are quickly removed, immersed in ice-cold, and oxygenated in Kreb's solution containing 113.1 mM NaCl, 4.6 mM KCl, 2.45 mM CaCl₂, 1.2 mM MgCl₂, 22.0 mM NaH₂PO₄, and 10.0 mM glucose; pH 7.4 with 95% O₂-5% CO₂. The ventricles are opened and papillary muscles are removed with chordae tandineae and a base of surrounding tissue intact. The tendinous ends of the muscles are ligated with silk thread, and the muscles are mounted in vertical, double-jacketed organ baths containing 10 mL of oxygenated Kreb's solution kept at 37° C. The tendinous end is attached to a Grass isometric force transducer, while a metal hook is inserted into the base of the muscle.

Following a 45-minute equilibration period under a 1 gram tension, control contractions are elicited by stimulating the muscle using stainless steel field electrodes at a frequency of 1.0 Hz, 2.0 ms duration. The amplitude of the stimulus is adjusted to be approximately 1.5 times the threshold amplitude sufficient to elicit a contraction of the tissues. Control contraction-relaxation cycles are recorded for 30 seconds continuously. Cumulative test drug concentrations are then injected directly into the bath while the tissue is being stimulated. Contraction-relaxation recordings are made continuously, for 30 seconds per test compound concentration. A series of washout contractions is recorded following a change of solution. Provided that the amplitude of contraction returns to that measured in control conditions, a single concentration of positive control is then tested on the tissue in the same manner as the test compound.

Contraction amplitude as well as the time courses of contraction and relaxation are quantified. All recordings are normalized against control values; statistical analysis of the results is made using t-tests or ANOVAs.

In Vitro Effect on Contractility

The effect of the compounds of the present when administered alone and in combination of 100 nM isoproterenol on isolated cardiomyocytes is tested in isolated ventricular myocytes from rabbit hearts. Isoproterenol, a potent β-adrenergic agonist, can produce large increases in cardiac contraction, calcium transient amplitude, and the rates of relaxation (acceleration of relaxation or lusitropic effect). The effects of Isoproterenol are then antagonized with different concentrations of a compound of the present invention.

Cardiac myocytes are digested from healthy white New Zealand male rabbits (3-5 lbs), with enzymatic digestion. Briefly, each animal is anesthetized with ketamine (50 mg/kg) and xylazine (6 mg/kg)-IM injection in hind limb. Once animal is sedated (˜10-15 min), 0.1-0.3 ml of pentobarbital is injected into the ear vein. The heart is exposed by a cut just below the rib-cage and bilateral thoracotomy and removed rapidly ensuring that aorta remains intact. The heart is immediately placed in oxygenated NT with Ca2+ placed on ice for rinsing the blood out, cleared from vessels and pericardium, cannulated and maintained at 37° C. The heart is retrogradedly perfused and tissue digested with collagenase and protease. Digested myocytes are subsequently stored in 0.1 mM Ca2+ normal tyrodes for further analyses. Sarcomere length changes are recorded at 37° C. in the presence of 2 mM calcium and analyzed with an IonOptix system. Sarcomere length data is acquired for each myocyte over an average of 10 beats duration, at pacing rates of 1, 2, and 3 Hz. Basal percent sarcomere shortening and length-frequency relation of each myocyte is evaluated, and serves as a measure of cellular viability.

In Vivo Effect on Contractility and β-Adrenergic Antagonist Activity

Studies were performed on White New Zealand male rabbits (2-3 kg weight). Animals were initially anesthetized with ketamine (50 mg/kg; IM) and xylazine (6 mg/kg, IM). Subsequently, animals were intubated (via tracheotomy; 3 mm tube) and ventilated with 2% isoflurane (mixed in 95% O2+5% CO2). Each rabbit was instrumented for LV pressure (3F Millar catheter) through right carotid artery, arterial pressure (3F catheter, Cook Instruments) through left femoral artery which was connected to a fluid-filled pressure transducer (BD Instruments), and ECG. Both pressure transducers were zeroed against atmospheric pressure, and calibrated before each study using an analog manometer. Upon completion of instrumentation, isoflurane was reduced to 1.25%, and the animal was covered for maintenance of body core temperature. Arterial and LV blood pressures (LVP) and the ECG signals were simultaneously digitally recorded on a PC. The recording system (Gould Instruments) and the corresponding software (Ponema, Gould Instruments) facilitates detailed calculation of various parameters directly from all signals and recorded on a separate file with a 1 sec resolution. Upon completion of the study protocol, rabbits were euthenized by isoflurane overdose (5% for at least 1 minute) and cardiac arrest through IV infusion of 5 ml 3 molar potassium chloride.

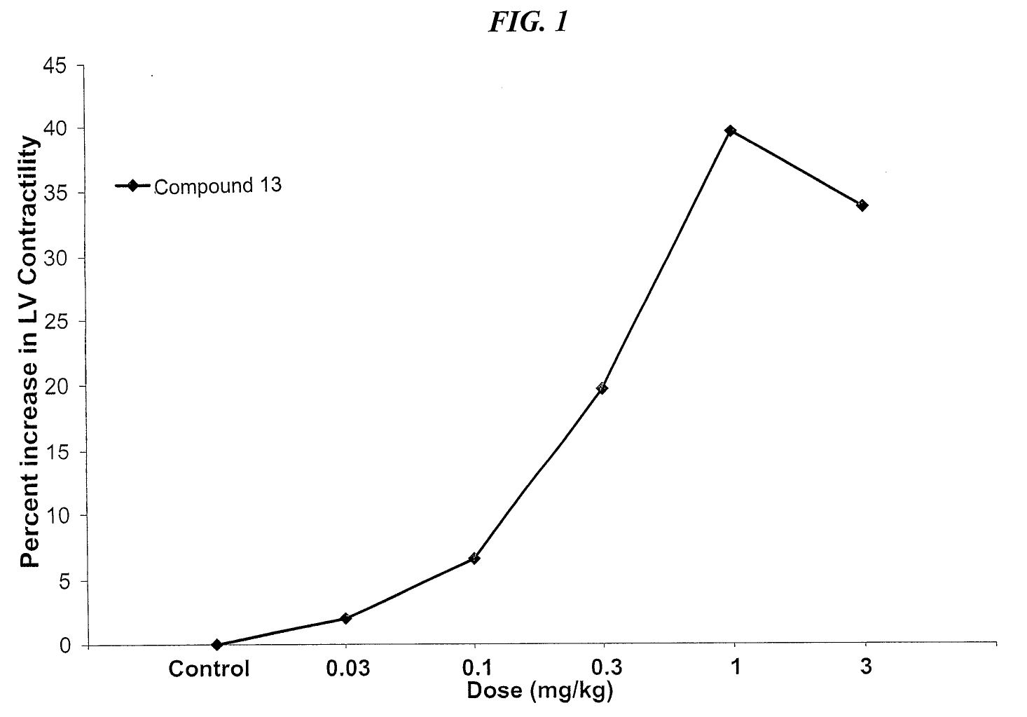

Once the physiological variables were stable, effects of each compound on hemodynamics and ECG were determined based on two different protocols: 1) infusion of a compound of the present invention to determine the effects of the compound itself mainly on contractility, and other hemodynamic indices; 2) infusion of a compound of the present invention while the system was challenged with 0.5 ug/kg isoproterenol to determine the beta-adrenergic antagonism properties of each tested compound. The results obtained from contractility studies performed using Compound 13 are shown in FIG. 1.

All publications, patents and patent applications identified above are herein incorporated by reference.

The invention being thus described, it will be apparent to those skilled in the art that the same may be varied in many ways without departing from the spirit and scope of the invention. Such variations are included within the scope of the invention to be claimed.

1. A compound of formula I
β-(Ar)ₙ-(L)ₘ-X (I)
or a pharmaceutically acceptable equivalent, an isomer or a mixture of isomers thereof, wherein:
m is 0 or 1;
n is 0 or 1;
β is a 2-amino-1-hydroxyeth-1-yl radical, an N-substituted-2-amino-1-hydroxyeth-1-yl radical, an N—N-disubstituted-2-amino-1-hydroxyeth-1-yl radical, a 3-amino-2-hydroxypropoxy radical, an N-substituted-3-amino-2-hydroxypropoxy radical, or an N—N-disubstituted-3-amino-2-hydroxypropoxy radicals;
Ar is, at each occurrence, the same or different, individually selected from aryl radicals and heteroaryl radicals, which aryl and heteroaryl radicals are unsubstituted or substituted with independently substituent(s) chosen from R₂, R₃, and R₄;
R₂, R₃, and R₄ are independently C₁-C₈ alkyl radicals, C₃-C₈ cycloalkyl radicals, C₂-C₈ alkenyl radicals, C₃-C₈ cycloalkenyl radicals, C₂-C₈ alkynyl radicals, C₃-C₈ cycloalkynyl radicals, C₁-C₄ alkylthio groups, C₁-C₄ alkoxy groups, halo radicals, a nitro group, a cyano group, a trifluoromethyl group, a trifluoroethyl group, a pentafluoroethyl group, a trifluoromethoxy group, —NR₅R₆ groups, acylaminoalkyl radicals, —NHSO₂R₁ groups or —NHCONHR₁ groups, wherein one or more —CH₂— group(s) of the alkyl, alkenyl and alkynyl radicals is/are optionally replaced with —O—, —S—, —SO—, —SO₂— and/or —NR₅—, and the alkyl, alkenyl and alkynyl radicals are unsubstituted or substituted with one or more substituent(s) chosen from an oxo group and a hydroxyl group;
R₅ and R₆ are independently a lone pair of electrons, a hydrogen radical, a C₁-C₈ alkyl radical, a C₂-C₈ alkenyl radical or a C₂-C₈ alkynyl radical, wherein the alkyl, alkenyl and alkynyl radicals are unsubstituted or substituted with a phenyl radical or a substituted phenyl radical;
R₁ is a hydrogen radical, a C₁-C₈ alkyl radical, a C₃-C₈ cycloalkyl radical, a C₂-C₈ alkenyl radical, a C₃-C₈ cycloalkenyl radical, a C₂-C₈ alkynyl radical or a C₃-C₈ cycloalkynyl radical;
L is a direct bond, a C₁-C₁₂ alkylene radical, a C₂-C₁₂ alkenylene radical or a C₂-C₁₂ alkynylene radical, wherein one or more —CH₂— group(s) of the alkylene, alkenylene and alkynylene radicals is/are optionally replaced with —O—, —S—, —SO₂—, —NR₅—, C₃-C₈ cycloalkylene and/or C₃-C₈ heterocycloalkylene, and the alkylene, alkenylene and alkynylene radicals are unsubstituted or substituted with one or more substituent(s) independently chosen from an oxo group and a hydroxyl group; and
X is a moiety of formula A, B, C, D, E, F, G, H, I, J, K, L, M, N, O, P, Q, R, S, T, U, V, W or Y 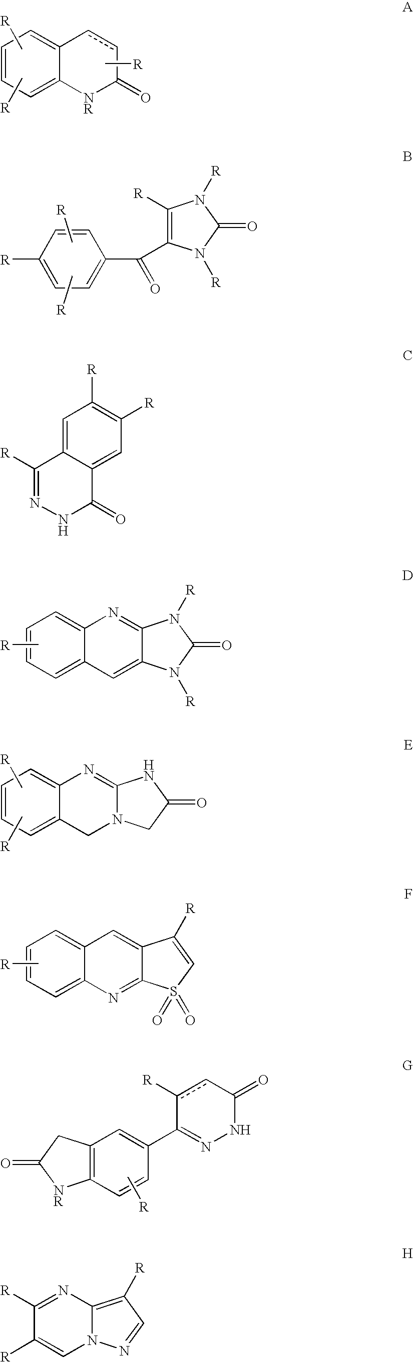 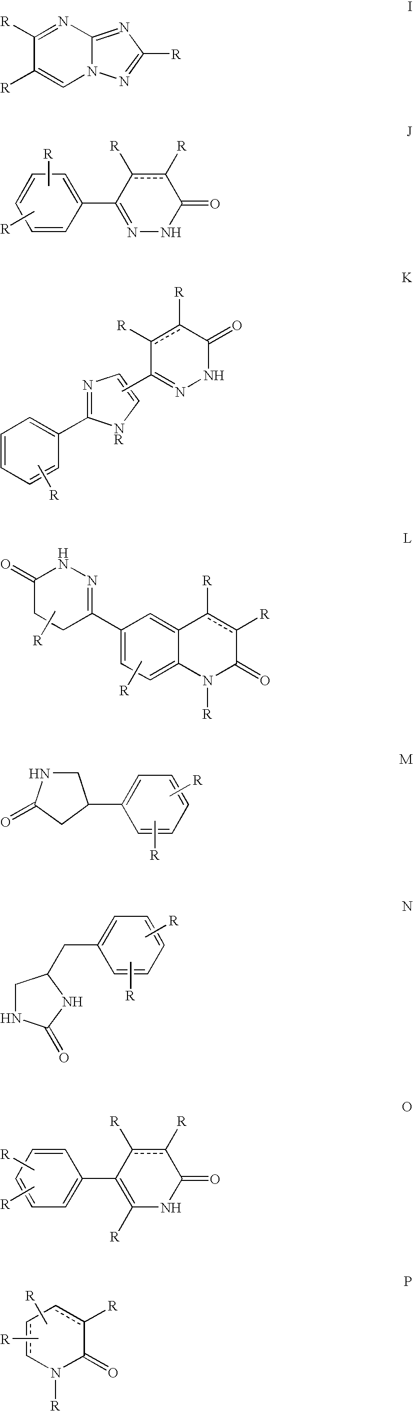 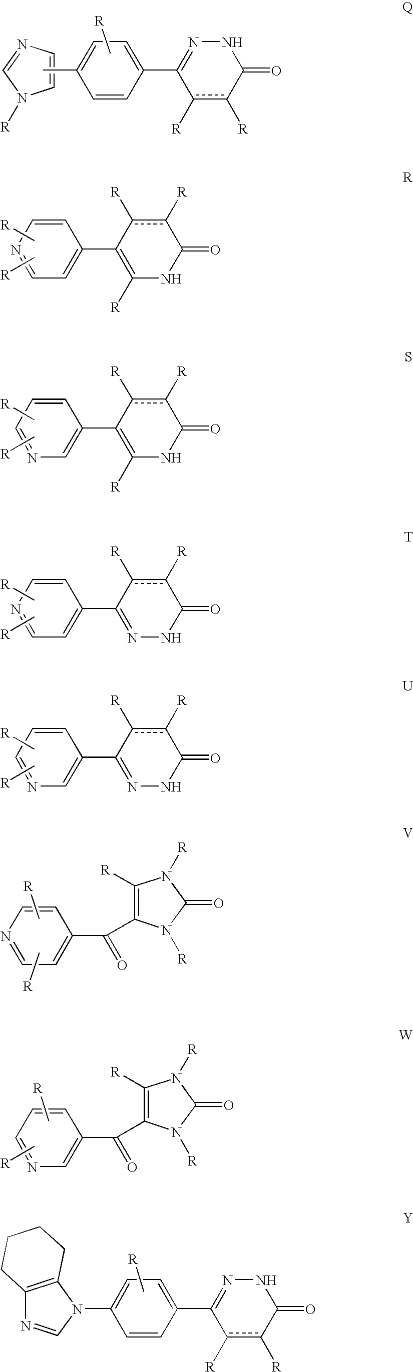
with X connected to L through any one R; and
wherein one R group of moieties A-Y forms a covalent bond between X and L when m is 1, or between X and Ar when n is 1 and m is 0, or between X and β when n is 0 and m is 0; and each remaining R group of moieties A-Y is independently chosen from a hydrogen radical, halo radicals, a nitro group, a cyano group, a trifluoromethyl group, an amino group, NR₅R₆ groups, C₁-C₄ alkoxy radicals, C₁-C₄ alkylthio radicals, COOR₁ radicals, C₁-C₁₂ alkyl radicals, C₂-C₁₂ alkenyl radicals and C₂-C₁₂ alkynyl radicals, wherein one or more —CH₂-group(s) of the alkyl, alkenyl and alkynyl radicals is/are optionally replaced with —O—, —S—, —SO₂— and/or —NR₅—, and the alkyl, alkenyl and alkynyl radicals are unsubstituted or substituted with one or more substituent(s) chosen from an oxo group and a hydroxyl group,
wherein said compound comprises one or more of the following:
(i) one or more of R2, R3, or R4 is selected from C₃-C₈ cycloalkyl radicals, C₃-C₈ cycloalkenyl radicals, C₃-C₈ cycloalkynyl radicals, a trifluoroethyl group, a pentafluoroethyl group, and a trifluoromethoxy group;
(ii) L is selected from C₃-C₈ cycloalkylene and/or C3-C8 heterocycloalkylene; and
(iii) X is selected from R, S, T, U, V, W and Y.

2. The compound of claim 1, wherein formula (I)'s Ar is each independently chosen from groups Ar₁, Ar₂, Ar₃, Ar₄, Ar₅, Ar₆ and Ar₇: 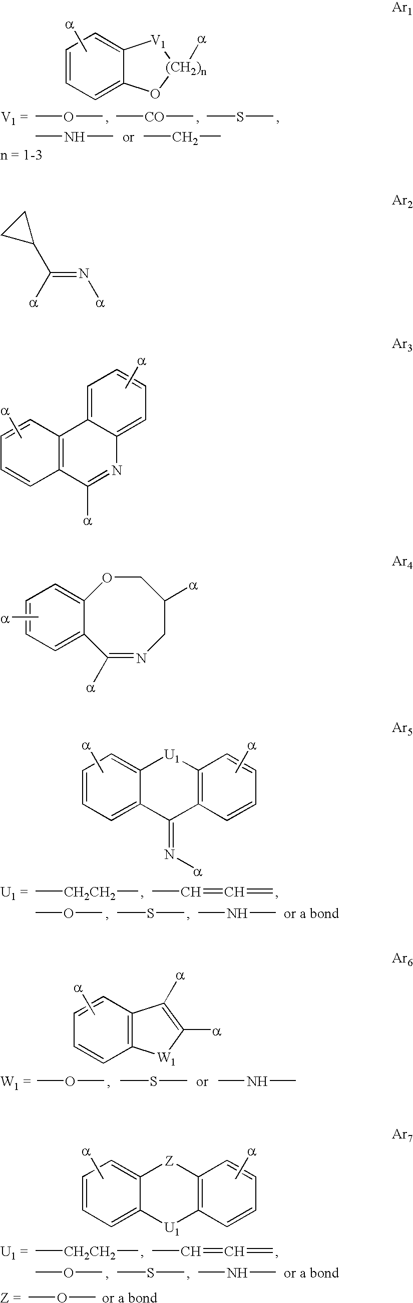
wherein α indicates the position where Ar may bond to β, L, and X.

3. The compound of claim 1, wherein X is chosen from moieties of formulas A, B, C, D, E, F, G, H, I, J, K, L, M, N, O, P, Q, R, S, T, U, V, W, and Y, wherein Ar is group Ar₇, and wherein Z is a bond.

4. The compound of claim 1, wherein formula (I)'s Ar is a phenyl radical.

5. The compound of claim 4, wherein the phenyl radical is unsubstituted.

6. The compound of claim 1, wherein formula (I)'s Ar is group Ar₇.

7. The compound of claim 6, wherein group Ar₇'s Z is a bond.

8. The compound of claim 6, wherein group Ar₇'s U₁ is —NH—.

9. The compound of claim 1, wherein one or more of formula (I)'s β, the N-substituted-2-amino-1-hydroxyeth-1-yl radicals, the N—N-disubstituted-2-amino-1-hydroxyeth-1-yl radicals, the N-substituted-3-amino-2-hydroxypropoxy radicals, or N—N-disubstituted-3-amino-2-hydroxypropoxy radicals are substituted with any group capable of bonding to such radicals.

10. The compound of claim 1, wherein formula (I)'s β is chosen from radicals of formula (β₁) and radicals of formula (β₂):
—CHOHCH₂NZ₁Z₂ (β₁); and
—OCH₂CHOHCH₂NZ₁Z₂ (β₂);
wherein Z₁ and Z₂ are independently chosen from a hydrogen radical, R₁ radicals, and —CH₂CH₂-yl-R₁ radicals; and wherein Y, is chosen from a —NHCO— radical, a —NHCONH— radical, and a —NHSO₂— radical.

11. The compound of claim 1, wherein formula (I)'s β is —OCH₂CHOHCH₂NZ₁Z₂.

12. The compound of claim 1, wherein formula (I)'s Z₁ and Z₂ are independently selected from a hydrogen radical and R₁ radicals.

13. The compound of claim 1, wherein Z₁ is hydrogen and Z₂ is C₁-C₄ alkyl.

14. The compound of claim 1, wherein Z₂ is isopropyl or tert-butyl.

15. The compound of claim 1, wherein formula (I)'s L is chosen from C₁-C₁₂ alkylene radicals, wherein one or more —CH₂— group(s) of the alkylene radicals is/are replaced with —O— and/or —NR₅—, and/or the alkylene radicals are substituted with one or more oxo group(s).

16. The compound of claim 1, wherein L is chosen from —(CH₂)ₚO(CH₂)_{q}O—, —(CH₂)ₚO—, —(CH₂)ₚNH(CO)(CH₂)_{q}O— and —(CH₂)ₚ(CO)NH(CH₂)_{q}NH(CO)(CH₂)ᵣO—, wherein p, q and r are independently 0, 1, 2, 3 or 4.

17. The compound of claim 1, wherein L is —(CH₂)ₚO(CH₂)_{q}O—, and wherein q is 1, 2, 3 or 4.

18. The compound of claim 17, wherein p is 0 or 1.

19. The compound of claim 18, wherein L is —O(CH₂)₃O— or —CH₂O(CH₂)₃O—.

20. The compound of claim 1, wherein L is —(CH₂)ₚO—, and wherein p is 1, 2, 3 or 4.

21. The compound of claim 20, wherein L is —(CH₂)₂O—.

22. The compound of claim 1, wherein L is —(CH₂)ₚNH(CO)(CH₂)_{q}O—, wherein p and q are independently 1, 2, 3 or 4. In further embodiments, p is 0 or 1. In yet further embodiments, L is —CH₂NH(CO)CH₂O— or —(CH₂)₂NH(CO)CH₂O—.

23. The compound of claim 1, wherein L is —(CH₂)ₚ(CO)NH(CH₂)_{q}NH(CO)(CH₂)ᵣO—, and wherein q and r are independently 1, 2, 3 or 4.

24. The compound of claim 23, wherein p is 0 or 1.

25. The compound of claim 23, wherein L is —(CO)NH(CH₂)₂NH(CO)CH₂O—, —CH₂(CO)NH(CH₂)₂NH(CO)CH₂O—, or —(CH₂)₂(CO)NH(CH₂)₂NH(CO)CH₂O—.

26. The compound of claim 1, wherein L is selected from the group consisting of: C₁-C₁₂ alkylene radicals, C₂-C₁₂ alkenylene radicals and C₂-C₁₂ alkynylene radicals, wherein one or more —CH₂— group(s) of the alkylene, alkenylene and alkynylene radicals is/are replaced with —C₃-C₈ cycloalkylene and/or C₃-C₈ heterocycloalkylene.

27. The compound of claim 1, wherein formula (I)'s X is chosen from the group consisting of moieties of formulas R, S and T, U, V, W and Y.

28. The compound of claim 1, wherein formula (I)'s X is chosen from moieties of formula S.

29. The compound of claim 1, wherein formula (I)'s X is chosen from moieties of formula J.

30. The compound of claim 1, wherein formula (I)'s R groups of moieties A-Y are independently chosen from the group consisting of: a hydrogen radical; C₁-C₁₂ alkyl radicals; C₂-C₁₂ alkenyl radicals; C₂-C₁₂ alkynyl radicals, halo radicals and cyano group.

31. The compound of claim 30, wherein formula (I)'s R groups of moieties A-Y are independently chosen from a hydrogen radical and halo radicals.

32. The compound of claim 31, wherein formula (I)'s R groups of moieties A-Y are independently chosen from a hydrogen radical and a chloro radical.

33. The compound of claim 1, wherein formula (I)'s R₁ is chosen from the group consisting of: a hydrogen radical, C₁-C₆ alkyl radicals, C₁-C₆ cycloalkyl radicals, C₂-C₆ alkenyl radicals, C₂-C₆ cycloalkenyl radicals, and C₂-C₆ alkynyl radicals.

34. The compound of claim 1, wherein formula (I)'s R₂, R₃ and R₄ are independently chosen from the group consisting of: a cyano group; a nitro group; halo radicals; a hydrogen radical; a trifluoromethyl group; acylaminoalkyl radicals, C₁-C₄ alkoxy groups; C₁-C₄ alkylthio groups; C₁-C₈ alkyl radicals; C₂-C₈ alkenyl radicals; and C₂-C₈ alkynyl radicals.

35. The compound of claim 34, wherein the acylaminoalkyl radicals contain an alkyl chain having from C₁-C₆.

36. The compound of claim 1, wherein formula (I)'s R₅ and R₆ are independently chosen from the group consisting of: a lone pair of electrons; a hydrogen radical; C₁-C₈ alkyl radicals; C₂-C₈ alkenyl radicals; and C₂-C₈ alkynyl radicals.

37. The compound of claim 1, wherein said compound is N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)phenyl]-acetamide (7).

38. The compound of claim 1, wherein said compound is N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylamino-propoxy)-phenyl]-acetamide (12b).

39. The compound of claim 1, wherein said compound is N-(2-{2-[2-Chloro-4-(6-oxo-1,4,5,6-tetrahydropyridazin-3-yl)-phenoxy]-acetylamino}-ethyl)-2-(4-((S)-2-hydroxy-3-isopropylaminopropoxy)-phenyl]-benzamide (12a).

40. The compound of claim 1, wherein said compound is 4-((S)-3-tert-Butylamino-2-hydroxy-propoxy)-N-(2-{2-[2-chloro-4-(6-oxo-1,4,5,6-tetrahydropyridazin-3-yl)-phenoxy]-acetylamino}-ethyl)-benzamide (13).

41. The compound of claim 1, wherein said compound is 2-[2-chloro-4-(6-oxo-1,4,5,6-tetrahydro-pyridazin-3-yl)-phenoxy]-N-[4-(2-hydroxy-3-isopropylaminopropoxy)-benzyl]-acetamide (17a).

42. The compound of claim 1, wherein said compound is N-[4-(3-tert-butylamino-2-hydroxy-propoxy)-benzyl]-2-[2-chloro-4-(6-oxo-1,4,5,6-tetrahydro-pyridazin-3-yl)-phenoxy]-acetamide (17b).

43. The compound of claim 1, wherein said compound is 2-[2-chloro-4-(6-oxo-1,4,5,6-tetrahydro-pyridazin-3-yl)-phenoxy]-N-{2-[4-(2-hydroxy-3-isopropylamino-propoxy)-phenyl]-ethyl}-acetamide (17c).

44. The compound of claim 1, wherein said compound is N-{2-[4-(3-tert-butylamino-2-hydroxy-propoxy)-phenyl]-ethyl}-2-[2-chloro-4-(6-oxo-1,4,5,6-tetrahydropyridazin-3-yl)-phenoxy]-acetamide (17d).

45. The compound of claim 1, wherein said compound is N-{2-[4-((S)-3-tert-Butylamino-2-hydroxy-propoxy)-phenyl]-ethyl}-2-[4-(6-oxo-1,4,5,6-tetrahydropyridazin-3-yl)-phenoxy]-acetamide (21).

46. The compound of claim 1, wherein said compound is 6-(3-Chloro-4-{3-[4-(2-hydroxy-3-isopropylamino-propoxy)-benzyloxy]-propoxy}-phenyl)-4,5-dihydro-2H-pyridazin-3-one (31a).

47. The compound of claim 1, wherein said compound is 6-(4-{3-[4-(3-tert-Butylamino-2-hydroxy-propoxy)-benzyloxy]-propoxy}-3-chloro-phenyl)-4,5-dihydro-2H-pyridazin-3-one (31b).

48. The compound of claim 1, wherein said compound is 6-(3-chloro-4-{2-[4-(2-hydroxy-3-isopropylamino-propoxy)-phenyl]-ethoxy}-phenyl)-4,5-dihydro-2H-pyridazin-3-one (37a).

49. The compound of claim 1, wherein said compound is 6-(4-{2-[4-(3-tert-butylamino-2-hydroxy-propoxy)-phenyl]-ethoxy}-3-chloro-phenyl)-4,5-dihydro-2H-pyridazin-3-one (37b).

50. The compound of claim 1, wherein said compound is 6-[3-Chloro-4-(3-{2-[4-((S)-2-hydroxy-3-isopropylamino-propoxy)-phenyl]-ethoxy}-propoxy)-phenyl]-4,5-dihydro-2H-pyridazin-3-one (46a).

51. The compound of claim 1, wherein said compound is 6-[4-(3-{2-[4-((S)-3-tert-Butylamino-2-hydroxy-propoxy)-phenyl]-ethoxy}-propoxy)-3-chloro-phenyl]-4,5-dihydro-2H-pyridazin-3-one (46b).

52. The compound of claim 1, wherein said compound is 2′{3-[4-(2-hydroxy-3-isopropylamino-propoxy)-phenoxy]-propoxy}-2-methyl-6-oxo-1,6-dihydro-[3,4′]bipyridinyl-5-carbonitrile.

53. The compound of claim 1, wherein said compound is 6′-{3-[4-2-hydroxy-3-isopropylamino-propoxy)-phenoxy]-propoxy}-2-methyl-6-oxo-1,6-dihydro-[3,3′]bipyridinyl-5-carbonitrile.

54. The compound of claim 1, wherein said compound is 6-[3-chloro-4-(2-{[4-(2-hydroxy-3-isopropylamino-propoxy)-9H-carbazol-1-yl]-methyl-amino]-ethoxy)-phenyl]-4,5-dihydro-2H-pyridazin-3-one.

55. The compound of claim 1, wherein said compound is 6-[4-(2-{[4-(3-tert-butylamino-2-hydroxy-propoxy)-9H-carbazol-1-yl]-methyl-amino}-ethoxy)-3-chloro-phenyl]-4,5-dihydro-2H-pyridazin-3-one.

56. The compound of claim 1, wherein said compound is 6-(3-chloro-4-{2-[(4-{2-hydroxyl-3-[2-(2-methoxy-phenoxy)-ethylamino]-propoxy}-9H-carbazol-1-yl)-methyl-amino]-ethoxy}-phenyl)-4,5-dihydro-2H-pyridazin-3-one.

57. A pharmaceutical composition comprising the compound of claim 1 and a pharmaceutically acceptable carrier.

58. The pharmaceutical composition of claim 57, wherein said composition is formulated for intravenous administration.

59. The pharmaceutical composition of claim 57, wherein said composition is formulated for oral administration.

60. A method of inhibiting β-adrenergic receptors and/or inhibiting phosphodiesterase, comprising administering an effective amount of the pharmaceutical composition of claim 57 to an animal in need of such treatment.

61. A method for regulating calcium homeostasis, comprising administering an effective amount of the pharmaceutical composition of claim 57 to an animal in need of such regulation.

62. A method for treating a disease, disorder or condition in which disregulation of calcium homeostasis is implicated, comprising administering an effective amount of the pharmaceutical composition of claim 57 to an animal in need of such treatment.

63. The method of claim 62, wherein said disease, disorder or condition is selected from the group consisting of: cardiovascular disease, stroke, epilepsy, an ophthalmic disorder, and a migraine.

64. The method of claim 63, wherein said cardiovascular disease is selected from the group consisting of: heart failure, hypertension, SA/AV node disturbance, arrhythmia, hypertrophic subaortic stenosis, angina, chronic heart failure, and congestive heart failure.

65. A method of treating congestive heart failure, comprising administering to a mammal in need of such treatment the pharmaceutical composition of claim 57.

66. A method of treating hypertension, comprising administering to a mammal in need of such treatment the pharmaceutical composition of claim 57.
